(19) United States
(12) Patent Application Publication
(10) Pub. No.: US 2010/0081713 A1
(43) Pub. Date: Apr. 1, 2010

(54) COMPOSITIONS AND METHODS FOR TREATING VIRAL INFECTIONS

(75) Inventors: Geeta Sharma, Singapore (SG); Ralf Altmeyer, Singapore (SG); Vishal Pendharker, Singapore (SG); Yu Chen, Singapore (SG); Michael Foley, Chestnut Hill, MA (US)

Correspondence Address: Gearhart Law LLC, 4 Femdale Avenue, Chatham, NJ, 07928, US

(73) Assignee: CombinatoRx, (Singapore) Pte. Ltd.

(21) Appl. No.: 12/406,716

(22) Filed: Mar. 18, 2009

Related U.S. Application Data
(60) Provisional application No. 61/069,917, filed on Mar. 19, 2008.

(51) Int. Cl. A61K 31/351; A61K 31/215; A61K 31/135; A61K 31/13; A61P 31/12

(52) U.S. Cl. 514/459; 514/529; 514/647; 514/662

(57) ABSTRACT

The present invention provides compositions, methods, and kits for treating or preventing a viral infection (e.g., an infection caused by an influenza virus).

### CROSS-REFERENCE TO RELATED APPLICATIONS

This application claims the benefit of U.S. Provisional Application No. 61/069,917, filed Mar. 19, 2008, which is hereby incorporated by reference.

### BACKGROUND OF THE INVENTION

The invention relates to treating viral infections such as influenza. Diseases caused by viruses are major health problems worldwide, and include many potentially fatal or disabilitating illnesses. Influenza virus, for example, affects 5-15% of the population during epidemics and causes upper respiratory tract infections. Hospitalization and deaths can occur, especially in high-risk groups (elderly, chronically ill and immuno-compromised). Between three and five million cases of severe influenza and between 250,000 and 500,000 deaths are recorded every year around the world. Accordingly, there exists a need for reducing influenza and other viral infections.

### SUMMARY OF THE INVENTION

We have identified agents and combinations of agents which reduce inflammatory response in cells infected with an influenza virus, and further, have shown that agents and combinations of agents can reduce mortality rates of mice infected with an influenza virus. On this basis, the present invention provides compositions, methods, and kits useful in treating viral infections such as influenza.

Accordingly, in a first aspect, the invention features a composition comprising a combination of agents listed in Table 1 (e.g., further in combination with an antiviral agent) or a combination of an SSRI with an antiviral agent. The SSRI may be cericlamine, citalopram, clovoxamine, cyanodothiepin, dapoxetine, escitalopram, femoxetine, fluoxetine, fluvoxamine, ifoxetine, indalpine, indeloxazine, litoxetine, milnacipran, paroxetine, sertraline, tametraline, viqualine, and zimeldine; or analog thereof. In certain embodiments, the SSRI is sertraline or an analog thereof. The antiviral agent may be a Group A antiviral agent (e.g., oseltamivir, zanamivir, amantadine, and rimantadine). The agents may be present in an amount sufficient to treat a viral infection (e.g., an influenza infection caused by any of the types, subtypes, or strains described herein). The composition may be formulated for administration by any route known in the art such as oral, parenteral (e.g., intravenously or intramuscularly), rectal, determatological, cutaneous, nasal, vaginal, inhalant, skin (patch), ocular, intrathecal, and intracranial. In certain embodiments the composition includes, consists of, or consists essentially of (a) a pair of agents shown in Table 1, or pharmaceutically acceptable salts thereof; and (b) a pharmaceutically acceptable carrier.

TABLE 1

Amoxapine + Dipyridamole
Amoxapine + Prednisolone
Budesonide + Nortriptyline (e.g., HCl)
Bufexamac + Prednisolone
CME-Amoxapine + Prednisolone
Desloratidine + Cyclosporine
Desloratidine + Fluoxetine
Desloratidine + Nortriptyline (e.g., HCl)
Dipyridamole + Budesonide
Dipyridamole + Ibudilast
Epinastine + Prednisolone
Nortriptyline (e.g., HCl) + Prednisolone
Paroxetine (e.g., HCl) + Prednisolone
Paroxetine (e.g., HCl) + Dipyridamole
Sertraline + Prednisolone

In another aspect, the invention features a method for treating or preventing a viral infection in a patient. The method includes administering to the subject an amount of an SSRI sufficient to treat or prevent the viral infection in the patient. In certain embodiments, the patient has not been diagnosed with or does not suffer from depression, major depressive disorder, obsessive-compulsive disorder, panic disorder, posttraumatic stress disorder, social anxiety disorder, generalized anxiety disorder, or premenstrual dysphoric disorder. The SSRI may be selected from the group consisting of cericlamine, citalopram, clovoxamine, cyanodothiepin, dapoxetine, escitalopram, femoxetine, fluoxetine, fluvoxamine, ifoxetine, indalpine, indeloxazine, litoxetine, milnacipran, paroxetine, sertraline, tametraline, viqualine, and zimeldine; or analog thereof.

In another aspect, the invention features a method for treating or preventing a viral infection in a patient. The method includes administering to the patient a combination of agents selected from the group consisting of:

(a) a tricyclic antidepressant (e.g., amoxapine, nortriptyline, or an analog thereof) and a corticosteroid (e.g., prednisolone, budesonide, or an analog thereof);

(b) an SSRI (e.g., sertraline, paroxetine, or an analog thereof) and a corticosteroid (e.g., prednisolone, or an analog thereof);

(c) a tricyclic antidepressant (e.g., amoxapine, nortriptyline, or an analog thereof) and a tetra-substituted pyrimidopyrimidine (e.g., dipyridamole, or an analog thereof);

(d) a corticosteroid (e.g., budesonide, or an analog thereof); and a tetra-substituted pyrimidopyrimidine (e.g., dipyridamole, or an analog thereof);

(e) an SSRI (e.g., paroxetine, or an analog thereof) and a tetrasubstituted pyrimidopyrimidine (e.g., dipyridamole, or an analog thereof);

(f) tetrasubstituted pyrimidopyrimidine (e.g., dipyridamole, or an analog thereof) and ibudilast, or analog thereof;

(g) an antihistamine (e.g., epinastine, or an analog thereof) and a corticosteroid (e.g., prednisolone, or an analog thereof);

(h) a corticosteroid (e.g., prednisolone, or an analog thereof) and bufexamac, or analog thereof;

(i) an antihistamine (e.g., desloratidine, or an analog thereof) and a non-steroidal immunophilin-dependent immunosuppressant (NsIDI) (e.g., a cyclosporine);

(j) a tricyclic antidepressant (e.g., nortriptyline, or an analog thereof) and an antihistamine (e.g., desloratidine, or an analog thereof); and

(k) an antihistamine (e.g., desloratidine, or an analog thereof) and an SSRI (e.g., fluoxetine, or an analog thereof);

where the two drugs are administered simultaneously or within 14 days of each other in amounts that together are sufficient to treat or prevent said viral infection in said patient. The SSRI may be selected, for example, from the group consisting of cericlamine, citalopram, clovoxamine, cyanodothiepin, dapoxetine, escitalopram, femoxetine, fluoxetine, fluvoxamine, ifoxetine, indalpine, indeloxazine, litoxetine, milnacipran, paroxetine, sertraline, tametraline, viqualine, and zimeldine; or analog thereof. The tricyclic antidepressant may be selected from the group consisting of maprotiline, amoxapine, 8-hydroxyamoxapine, 7-hydroxyamoxapine, loxapine, loxapine succinate, loxapine hydrochloride, 8-hydroxyloxapine, amitriptyline, clomipramine, doxepin, imipramine, trimipramine, desipramine, nortriptyline, and protriptyline; or an analog thereof.

In certain embodiments, the combination of agents is selected from the combinations of Table 1, or a combination including one or more analogs of the agents of Table 1. An agent may be administered by any route known in the art such as oral, parenteral (e.g., intravenously or intramuscularly), rectal, determatological, cutaneous, nasal, vaginal, inhalant, skin (patch), ocular, intrathecal, and intracranial. In any of the above methods, the two agents may be administered within 10, 7, 5, 4, 3, or 2 days, or within 24, 12, 6, 3, 2, or 1 hour).

In either of the above methods, the method may further include administration of additional antiviral therapy (e.g., a Group A antiviral agent). The additional antiviral therapy may be administered by any of routes described herein and may be administered within 14 days (e.g., within 10, 7, 5, 4, 3, 2, 1 days or within 12, 6, 3, 2, or 1 hours) of one or both of agents.

In either of the above methods, if the SSRI is sertraline, it may be administered in doses of at least 1, 2, 5, 10, 20, 30, 40, 50, 60, 75, 100, 150, or 200 mg/kg/day.

The invention also features kits. One kit includes (a) an SSRI; and (b) instructions for administering the SSRI to a patient for treating or preventing a viral infection.

Another kit includes (a) a tricyclic antidepressant; (b) a corticosteroid; and (c) instructions for administering (a) and (b) to a patient for treating or preventing a viral infection.

Another kit includes (a) an SSRI; (b) a corticosteroid; and (c) instructions for administering (a) and (b) to a patient for treating or preventing a viral infection.

Yet another kit includes (a) a tricyclic antidepressant, a corticosteroid, an SSRI, or ibudliast, or an analog thereof; (b) a tetrasubstitutied pyrimidopyrimide; and (c) instructions for administering (a) and (b) to a patient for treating or preventing a viral infection.

Another kit includes (a) an antihistamine; (b) a corticosteroid, an NsIDI, a tricyclic antidepressant, or an SSRI; and (c) instructions for administering (a) and (b) to a patient for treating or preventing a viral infection.

Another kit includes (a) bufexamac, or an analog thereof; (b) a corticosteroid; and (c) instructions for administering (a) and (b) to a patient for treating or preventing a viral infection.

In the above kits containing two agents, the agents may be present in a single composition or in separate compositions.

Another kit includes (a) a tricyclic antidepressant; and (b) instructions for administering (a) with at least one of a corticosteroid, a tetrasubstituted pyrimidopyrmidine, and an antihistamine to a patient for treating or preventing a viral infection.

Another kit includes (a) an SSRI; and (b) instructions for administering (a) with at least one of a corticosteroid, a tetrasubstituted pyrimidopyrmidine, and an antihistamine to a patient for treating or preventing a viral infection.

Another kit includes (a) a corticosteroid; and (b) instructions for administering (a) with at least one of a tricyclic antidepressant, a tetrasubstituted pyrimidopyrmidine, an antihistamine, an SSRI, and bufexamac or an analog thereof to a patient for treating or preventing a viral infection.

Another kit includes (a) a tetrasubstituted pyrimidopyrmidine; and (b) instructions for administering (a) with at least one of a corticosteroid, a tricyclic antidepressant, an SSRI, and ibudilast or an analog thereof to a patient for treating or preventing a viral infection.

Another kit includes (a) an antihistamine; and (b) instructions for administering (a) with at least one of a corticosteroid, an NsIDI, a tricyclic antidepressant, and an SSRI to a patient for treating or preventing a viral infection.

Another kit includes (a) ibudilast or an analog thereof; and (b) instructions for administering (a) with a tetrasubstituted pyrimidopyrmidine to a patient for treating or preventing a viral infection.

Another kit includes (a) bufexamac, or an analog thereof; and (b) instructions for administering (a) with a corticosteroid to a patient for treating or preventing a viral infection.

Yet another kit includes (a) an NsIDI, or an analog thereof; and (b) instructions for administering (a) with an antihistamine to a patient for treating or preventing a viral infection.

Another kit includes (a) a combination of agents selected from Table 1; and (b) instructions for administering (a) with a Group A antiviral agent to a patient for treating or preventing a viral infection.

Another kit includes (a) a Group A antiviral agent; and (b) instructions for administering (a) with an SSRI or with a combination of agents selected from Table 1 to a patient for treating or preventing a viral infection.

Another kit includes (a) an SSRI; (b) a Group A antiviral agent; and (c) instructions for administering (a) and (b) to a patient for treating or preventing a viral infection.

Another kit includes (a) an SSRI; and (b) instructions from administering (a) to a patient with a Group A antiviral for treating or preventing a viral infection.

The above kits may include a composition including a combination of agents listed in Table 1, or an analog of such an agent.

In any of the above embodiments, the viral infection may be caused by a virus of a family selected from the group consisting of orthomyxoviridae, adenoviridae, paramyxoviridae, and coronaviridae (e.g., an influenza virus or any virus described herein).

To “treat” is meant to administer one or more agents to measurably slow or stop the replication of a virus in vitro or in vivo, to measurably decrease the load of a virus (e.g., any virus described herein including an influenza virus) in a cell in vitro or in vivo, or to reduce at least one symptom (e.g., inflammation) associated with having a viral infection in a patient. Desirably, the slowing in replication, the decrease in viral load, or reduction in the symptom is at least 20%, 30%, 50%, 70%, 80%, 90%, 95%, or 99%, as determined using a suitable assay (e.g., a inflammation assay described herein) as compared to in the absence of the agent.

To “prevent” a disease is meant to reduce to frequency of appearance of the disease in a population of patients, the likelihood of an individual patient developing the disease, or to reduce the symptoms or severity of a disease upon its appearance by administering one or more agents to a patient prior to diagnosis of the disease or manifestation of disease symptoms.

By “an effective amount” is meant the amount of a agent, alone or in combination with another therapeutic regimen, required to treat a patient with a viral infection (e.g., caused by any virus described herein including an influenza virus) in a clinically relevant manner. A sufficient amount of an agent used to practice the present invention for therapeutic treatment of conditions caused by a virus varies depending upon the manner of administration, the age, body weight, and general health of the patient. Ultimately, the prescribers will decide the appropriate amount and dosage regimen. Additionally, an effective amount may be an amount of an agent in a combination of the invention that is safe and efficacious in the treatment of a patient having a viral infection over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

By a “low dosage” is meant at least 5% less (e.g., at least 10%, 20%, 50%, 80%, 90%, or even 95%) than the lowest standard recommended dosage of a particular agent formulated for a given route of administration for treatment of any human disease or condition. For example, a low dosage of an agent that treats a viral infection and that is formulated for administration by intravenous injection will differ from a low dosage of the same agent formulated for oral administration.

By a “high dosage” is meant at least 5% (e.g., at least 10%, 20%, 50%, 100%, 200%, 300%, 500%, 1,000%, 2,000%, 5,000%, or 10,000%) more than the highest standard recommended dosage of a particular agent for treatment of any human disease or condition.

By a “Group A Antiviral” is meant any of the compounds listed in Table 2.

TABLE 2

(+)-Calanolide A
(+)-Dihydrocalanolide A
145U87
2′-C-methyl-7-deaza-adenosine
2′-C-Methylcytidine
2-Nor-cyclic GMP
3,4-Dicaffeoylquinic acid
3-Hydroxymethyl dicamphanoyl
khellactone
3-Hydroxyphthaloyl-beta-
lactoglobulin
3-Nitrosobenzamide
4-Azidothymidine
4-Methyl dicamphanoyl
khellactone
524C79
739W94
A 160621
A 315675
A 315677
A 5021
A 74259
A 74704
A 77003
A 80735
A 80987
A 91883A
A 98881
A-837093
Abacavir
AC 2
Acemannan
Acetylcysteine-Zambon
ACH 126445
ACH 126447
Aciclovir (e.g., extended release,
controlled release, topical patch)
Aciclovir-PMPA
ACP HIP
Actinohivin
AD 439
AD 519
Adamantylamide dipeptide
Adefovir (e.g., dipivoxil)
ADS J1
Afovirsen
AG 1284
AG 1350
AG 1478
AG 1859
AG 555
AG 6840
AG 6863
AG-021541
AGT-1
AHA 008
Aidfarel
AL 721
Alamifovir
Albuferon
Albumin/interferon-alpha
Aldesleukin
ALN RSV01
Alovudine
Alpha HGA
Alpha-1PDX
Alpha-antitrypsin
Alvircept sudotox
Alvocidib
ALX 0019
ALX 404C
AM 285
AM 365
Amantadine
AMD 070
AMD 3329
AMD 3465
AMD 8664
Amdoxovir
Amidinomycin
Aminopeptidase
Amitivir
Ampligen
Amprenavir
AMZ 0026
ANA 971
ANA 975
Ancriviroc
Andrographis
Anti-CCR5 monoclonal antibody
Anti-CCR5/CXCR4 sheep
monoclonal antibody
Anti-CD3 monoclonal antibody
CD4IgG conjugate
Anti-CD4 monoclonal antibody
Anti-CD7 monoclonal antibody
Anti-CD8 monoclonal antibody
Anti-CMV monoclonal antibody
Anti-hepatitis B ribozyme
Anti-HIV catalytic antibody
Anti-HIV immunotoxin (IVAX)
Anti-HIV-1 human monoclonal
antibody 2F5
Anti-HIV-1 human monoclonal
antibody 2G12
Anti-HIV-1 human monoclonal
antibody 4E10
Antineoplaston AS2 1 (e.g., oral)
Anti-RSV antibody (Intracel,
Corp.)
Antisense oligonucleotide PB2
AUG
Aop-RANTES
Aphidicolin
Aplaviroc
Apricitabine
AQ 148
AR 132
AR 177
ARB 95214
ARB 97265
ARB 97268
Arbidol
ARQ 323
Artemether
Artemisinin
Artesunate
AS 101
AT 61
Atazanavir
Atevirdine
Atorvastatin
AV 1101
AV 2921
AV 2923
AV 2925
AV 2927
Avarol
AVI 4065
AVR 118
AXD 455
Azidodideoxyguanosine
Azodicarbonamide
Bafilomycin A1
Baicalin
Bavituximab
BAY 414109
BAY 439695
BAY 504798
BAY Z 4305
BB 10010
BB 2116
BCH 10652
BCH 371
BCH 527
BCTP
BCX 140
BCX 1591
BCX 1827
BCX 1898
BCX 1923
BEA
BEA 005
Bellenamine
Benanomicin A
Benzalkonium (e.g., chloride)
Benzalkonium
chloride/octoxynol 9 (e.g.,
vaginal gel)
Beta-D-FDOC
Beta-L-ddC
Beta-L-FddC
Bevirimat
BG 777
BGP 15
BILA 2185 BS
BILN 303 SE
BILR 355
BIRM ECA 10-142
BIVN 401
BL 1743
BLX 833 (e.g., controlled
release)
BM 510836
BMS 181167-02
BMS 181184
BMS 182193
BMS 186318
BMS 187071
BMS 488043
BMS 806
BMY 27709
Boceprevir (SCH 503034)
Brecanavir
Brefeldin A
Brequinar
Brivudine
BRL 47923DP
BSL 4
BST 5001
BTA 188
BTA 798
C 1605
C 2507
C31G
Calcium spirulan
Canventol
Capravirine
Carbendazim
Carbocyclic deazaadenosine
Carbopol polymer gel
Carbovir
CC 3052
CD4 fusion toxin
CD4 IgG
CD4-ricin chain A
Celgosivir
CellCept
Cellulose sulfate
Cepharanthine
Ceplene
CF 1743
CFY 196
CGA 137053
CGP 35269
CGP 49689
CGP 53437
CGP 53820
CGP 57813
CGP 61783
CGP 64222
CGP 70726
CGP 75136
CGP 75176
CGP 75355
Chloroquine (e.g., phosphate)
CI 1012
CI 1013
Cidofovir
Ciluprevir (BILN 2061)
Civacir
Civamide
CL 190038
CL 387626
Clevudine
CMV 423
CMX 001
CNBA-Na
CNJ I02
Cobra venom peptide
Colloidal silver
Conocurvone
Cosalane
Costatolide
CP 1018161
CP 38
CP 51
CPFDD
CpG 10101
CRL 1072
Crofelemer
CS 8958
CS 92
CT 2576
CTC 96
Curcumin
Curdlan sulfate
Cyanovirin-N
Cyclosporine
CYT 99007
Cytarabine
Cytomegalovirus immune
globulin
DAB486interleukin-2
DABO 1220
Dacopafant
DAP 30
DAP 32
Dapivirine
Darunavir
D-aspartic-beta-hydroxamate
DB 340
DDCDP-DG
DDGA
Deazaadenosine
Deazaneplanocin A
DEB 025
DEBIO-025
Delavirdine
Delmitide
Denileukin diftitox
Deoxyfluoroguanosine
DES 6
Dexelvucitabine
Dextran sulfate
Dextrin 2-sulfate
DG 35
Didanosine
Dideoxyadenosine
Dideoxyguanosine
Dideoxythymidine
Didox
Dihydroartemisinin
Dihydrocostatolide
Dinitrochlorobenzene
DL 110
DMP 323
DMP 850
DMP 851
DmTr-ODN12
Docosanol
DP 107
DPC 082
DPC 083
DPC 681
DPC 684
DPC 961
DPC 963
Droxinavir
DUP 925
DYE
E 913
EB-Foscarnet
Edodekin alfa
Edoxudine
E-EPSEU
Efavirenz
EGS 21
EHC 18
EHT 899
Elvucitabine
EM 1421
EM 2487
Emivirine
Emtricitabine
Emtricitabine/tenofovir
disoproxil fumarate
EMZ 702
Enfuvirtide
Entecavir
Eosinophil-derived neutralizing
agent
Episiastatin B
ET 007
Etanercept
Ether lipid analogue
Etoviram
Etravirine
F 105
F 36
F 50003
Famciclovir
Fas-ligand inhibitor
Fasudil
Fattiviracin A1
FEAU
Feglymycin
Felvizumab
FGI 345
Fiacitabine
Fialuridine
FLG
Floxuridine
Flutimide
Fluvastatin (e.g., sodium)
Fomivirsen
Fosalvudine tidoxil
Fosamprenavir
Foscarnet Sodium
Fozivudine
FP 21399
F-PBT
FPMPA
FPMPDAP
FR 191512
FR 198248
Galactan sulfate
Ganciclovir
GAP 31
GCA 186
GCPK
GE 20372A
GE 20372B
GEM 122
GEM 132
GEM 144
GEM 92
GEM 93
Gemcitabine (e.g.,
hydrochloride)
Ginseng
Glamolec
Glutathionarsenoxide
Glycovir
Glycyrrhizin
GMDP
GO 6976
GO 7716
GO 7775
Gossypol
GPG-NH2
GPI 1485
GPI 2A
GPs 0193
GR 137615
GR 92938X
GS 2838
GS 2992
GS 3333
GS 3435
GS 4071
GS 438
GS 7340
GS 9005
GS 9132
GS 9160
GS 930
GW 275175
GW 5950X
HB 19
HBY 946
HCV 086
HCV 371
HCV AB 68
HCV-796
HCV-SM
HE 2000
HE 317
Hepatitis B immune globulin
Hepatitis C immune globulin
Hepex C
HEPT
Heptazyme
HGS-H/A27
HI 236
HI 240
HI 244
HI 280
HI 346
HI 443
HI 445
Histamine
Histamine dihydrochloride (e.g.,
injection, oral)
HIV DNA vaccine (Antigen
Express, Inc.)
HIV immune globulin
HIV immune plasma
HL 9
HOE BAY 793
HRG 214
HS 058
HuMax-HepC
Hydroxycarbamide
Hydroxychloroquine
Hypericin
I 152
IAZT
ICN 17261
IDN 6556
Idoxuridine
IM28
Imiquimod
ImmStat
ImmuDyn
Immunocal
Imreg 1
Incadronic acid
INCB 9471
Indinavir
Infliximab
Influenza matrix protein Zn2+
finger peptide
Ingenol Triacetate
Inophyllum B
Inosine pranohex
Interferon
Interferon Alfa-2a
Interferon alfa-2b (e.g.,
inhalation)
Interferon alfacon-1
Interferon alpha (e.g., sustained
release, intranasal, Omniferon)
Interferon alpha-2b (e.g.,
controlled release or tranadermal)
Interferon alpha-2b gene therapy
Interferon alpha-n3
Interferon beta-1a
Interferon beta-1b
Interferon gamma-1b
Interferon omega
Interferon-tau
Interleukin 10 (e.g., human
recombinant)
Interleukin-1 receptor type I
Interleukin-13
Interleukin-15
Interleukin-16
Interleukin-2 agonist
Interleukin-4
IPdR
Ipilimumab
Isatoribine
ISIS 13312
ISIS 14803
Iso ddA
ITI 002
ITI 011
ITMN-191
JBP 485
JCA 304
JE 2147
JM 1596
JM 2763
JTK 003
JTK 109
JTK 303
K 12
K 37
K 42
Kamizol
kethoxal
Kijimicin
Kistamicin
KKKI 538
KM 043
KNI 102
KNI 241
KNI 272
KNI 413
KNI 684
Kootikuppala
KP 1461
KPC 2
KPE 00001113
KPE 02003002
KRH 1120
L 689502
L 693549
L 696229
L 696474
L 696661
L 697639
L 697661
L 708906
L 731988
L 732801
L 734005
L 735882
L 738372
L 738684
L 738872
L 739594
L 748496
L 754394
L 756423
L 870810
L HSA ara AMP
Lactoferrin
Lamivudine
Lamivudine/abacavir
Lamivudine/zidovudine
Lamivudine/zidovudine/abacavir
Lasinavir
LB 71116
LB 71148
LB 71262
LB 71350
LB 80380
LB 84451
L-chicoric acid
Lecithinized superoxide
dismutase
Leflunomide
Lentinan
Leukocyte interleukin injection
(CEL-SCI Corp.)
Leukotriene B4-LTB4
Levcycloserine
Levofloxacin
Lexithromycin
Licorice root
Liposomal ODG-PFA-OMe
Lithium succinate
Lobucavir
Lodenosine
Lopinavir
Lovastatin
Loviride
Lufironil
LY 180299
LY 214624
LY 253963
LY 289612
LY 296242
LY 296416
LY 309391
LY 309840
LY 311912
LY 314163
LY 314177
LY 316683
LY 326188
LY 326594
LY 326620
LY 338387
LY 343814
LY 354400
LY 355455
LY 366094
LY 366405
LY 368177
LY 73497
Lysozyme
M 40401
M4N
Madu
Mannan sulfate
MAP 30
Maraviroc
Maribavir
Masoprocol
MB-Foscarnet
MC 207044
MC 207685
MC 867
mCDS71
MDI-P
MDL 101028
MDL 20610
MDL 27393
MDL 73669
MDL 74428
MDL 74695
MDL 74968
MDX 240
ME 3738
ME 609
MEDI 488
Medusa Interferon
MEN 10690
MEN 10979
MER N5075A
Merimepodib (VX-497)
Met-enkephalin
Methisazone
Mevastatin
MGN 3
Michellamine B
Miglustat
Milk thistle
Mitoquinone
MIV 150
MIV 210
Mivotilate
MK 0518
MK 944A
MM 1
MMS 1
MOL 0275
Monoclonal antibody 1F7
Monoclonal antibody 2F5
Monoclonal antibody 3F12
Monoclonal antibody 447-52D
Monoclonal antibody 50-61A
Monoclonal antibody B4
Monoclonal antibody HNK20
Monoclonal antibody NM01
Mopyridone
Moroxydine
Motavizumab
Motexafin gadolinium
Mozenavir
MPC 531
MRK 1
MS 1060
MS 1126
MS 8209
MS 888
MSC 127
MSH 143
MTCH 24
MTP-PE
Murabutide
MV 026048
MX 1313
Mycophenolate mofetil
Mycophenolic Acid
Navuridine
NB 001
Nelfinavir (e.g., mesylate)
Neomycin B-arginine conjugate
Neotripterifordin
Nevirapine
NIM 811
Nitazoxanide
Nitric oxide (e.g., ProStrakan)
Nitrodeazauridine
NM 01
NM 49
NM 55
N-nonyl-DNJ
NNY-RANTES
Nonakine
NOV 205
NP 06
NP 77A
NPC 15437
NSC 158393
NSC 20625
NSC 287474
NSC 4493
NSC 615985
NSC 620055
NSC 624151
NSC 624321
NSC 627708
NSC 651016
NSC 667952
NSC 708199
NV 01
NV-08
Octoxynol 9
OCX 0191
OH 1
OKU 40
OKU 41
Oltipraz
Omaciclovir
Opaviraline
OPT TL3
Oragen
ORI 9020
Oseltamivir
Oxetanocin
Oxothiazolidine carboxylate
P 56
PA 344/PA 344B
Palinavir
Palivizumab
PAMBAEEG
Papuamide A
PBS 119
PC 1250
PC 515
PCL 016
PD 0084430
PD 144795
PD 153103
PD 157945
PD 169277
PD 171277
PD 171791
PD 173606
PD 173638
PD 177298
PD 178390
PD 178392
PD 190497
Pegaldesleukin
Peginterferon alfa-2a
Peginterferon alfa-2b
PEGinterferon alfacon-1
PEGylated interferon
Pegylated thymalfasin
Peldesine
PEN 203
Penciclovir
Pentosan polysulfate
Pentoxifylline
Peptide T
Peramivir
PETT 4
PF-03491390
PG 301029
PG 36
Phellodendrine
Phosphatidyllamivudine
Phosphatidylzalcitabine
Phosphatidylzidovudine
Phosphazid
Phosphinic cyclocreatine
Pinosylvin
Pirodavir
PL 2500
Pleconaril
Plerixafor
PM 104
PM 19
PM 523
PM 92131
PM 94116
PMEDAP
PMS 601
PMTG
PMTI
PN 355
PNU 103657
PNU 142721
podophyllotoxin
Poly ICLC
Polyadenylic polyuridylic acid
Polysaccharide K
PP 29
PPB 2
PPL 100
Pradefovir
Pradimicin A
Prasterone
PRO 140
PRO 2000
PRO 367
PRO 542
Probucol (Vyrex Corp.)
Propagermanium
Prostratin
Pseudohypericin
PSI 5004
PSI-6130
PTPR
PTX 111
Pyriferone
Q 8045
QM 96521
QM 96639
QR 435
Quinobene
Quinoxapeptin A
Quinoxapeptin B
QYL 438
QYL 609
QYL 685
QYL 769
R 1518
R 1626
R 170591
R 18893
R 61837
R 71762
R 803
R 82150
R 82913
R 851
R 87366
R 91767
R 944
R 95288
R-1626
R7128
Raluridine
Ramatroban
Ranpirnase
RB 2121
RBC CD4
RD 30028
RD 42024
RD 42138
RD 42217
RD 42227
RD 62198
RD 65071
RD6 Y664
Regavirumab
Resiquimod
Resobene
Respiratory syncytial virus
immune globulin
Retrogen
REV 123
RFI 641
Ribavirin
Rilpivirine
Rimantadine
Ritonavir
RKS 1443
RO 0334649
RO 247429
RO 250236
RO 316840
RO 53335
Robustaflavone
Rolipram
Rosiglitazone
RP 70034
RP 71955
RPI 312
RPI 856
RPR 103611
RPR 106868
RPR 111423
RS 654
RS 980
RSV 604
Rubitecan
Rupintrivir
S 1360
S 2720
S 9a
SA 1042
SA 8443
Saquinavir (e.g., mesylate)
Sargramostim
SB 180922
SB 205700
SB 206343
SB 73
SC 49483
SC 55099
SCH 350634
SCH 6
Schisandra
SCV 07
SCY-635
SD 894
S-DABO
SDF 1
SDZ 282870
SDZ 283053
SDZ 283471
SDZ 89104
SDZ PRI 053
SE 063
Semapimod
Sevirumab
SF 950
SF 953
Siamycin 1
Siamycin 2
sICAM-1
Sifuvirtide
SIGA 246
Silipide
Simvastatin
Simvastatin hydroxy acid,
ammonium salt
Sizofiran
SJ 3366
SK 034
SKF 108922
SKI 1695
SO 324
Sodium laurilsulfate
Solutein
Sorivudine (e.g., topical)
SP 10
SP 1093V
Sparfosic acid
SPC 3
SPD 756
SpecifEx-Hep B
SPI 119
SPL 2992
SPL 7013
SPV 30
SR 10204
SR 10208
SR 11335
SR 3745A
SR 3773
SR 3775
SR 3784
SR 3785
SR 41476
SRL 172
SRR SB3
ST 135647
Stachyflin
stallimycin
Stampidine
Statolon
Stavudine
Stepronin
Suksdorfin
Sulfated maltoheptaose
Superoxide dismutase
Suramin (e.g., sodium)
Sy 801
T 1100
T 118
T 22
T 30695
T 611
T 705
T4GEN
Tacrine
TAK 220
TAK 652
TAK 779
Talviraline
TAP 29
Taribavirin
TASP
Teceleukin
Tecogalan (e.g., sodium)
TEI 2306
Telaprevir (VX-950)
Telbivudine
Telinavir
Temacrazine
Tenidap
Tenofovir
Tenofovir disoproxil fumarate
TGG II 23A
TH 9407
TH 9411
Thalidomide
Thiophosphonoformic acid
Thiovir
Thymalfasin (e.g., Zadaxin)
Thymoctonan
Thymosin fraction 5
Thymotrinan
Thymus extract
tICAM-1
Tifuvirtide
Tilarginine
Tipranavir
Tiviciclovir
Tivirapine
TJ 41
TJ 9
TL 3024
TMC 126
TNF-alpha inhibitor
TNK 6123
TNX 355
Todoxin
TOFA
Tomeglovir
Transforming growth factor-
alpha
TraT
Trecovirsen
Tremacamra
Trichosanthin
Triciribine
Triconal
Trifluridine
Trimidox
Trodusquemine
Tromantadine
Trovirdine
Tucaresol
Tunicamycin
Tuvirumab
U 103017
U 75875
U 78036
U 80493
U 81749
U 88204E
U 96988
U 9843
UA 926
Ubenimex
UC 10
UC 16
UC 38
UC 42
UC 68
UC 70
UC 781
UC 81
UC 82
UIC 94003
Ukrain
UL36ANTI
UMJD 828
Ursodeoxycholic acid
UT 231B
Valaciclovir
Valganciclovir
Valomaciclovir
Valopicitabine (NM 283)
Valopicitabine (NM-283)
Valtorcitabine
Varicella zoster immune globulin
VB 19038
Vesnarinone
VF 1634
VGV 1
VGX 410
Vicriviroc
Vidarabine
Vincristine (e.g., sulfate)
VIR 101
Viraprexin
Virodene
Virostat
Viscum album extract
VP 50406
VRT 21493
VRX 496
VX 10166
VX 10217
VX 10493
VX 11106
WF 10
WHI 05
WHI 07
WIN 49569
WIN 49611
WM 5
WR 151327
XK 216
XK 234
XN 482
XP 951
XQ 9302
XR 835
XTL 2125
XTL 6865
XU 348
XU 430
Y-ART-3
YHI 1
YK FH312
Z 100
Z 15
Zalcitabine
Zanamivir
Zidovudine (e.g., phosphate-
didanosine dimer)
Zidovudine triphosphate mimics
ZX 0610
ZX 0620
ZX 0791
ZX 0792
ZX 0793
ZX 0851
ZY II

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the agents of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds useful in the invention include those described herein in any of their pharmaceutically acceptable forms, including isomers such as diastereomers and enantiomers, salts, solvates, and polymorphs thereof, as well as racemic mixtures. Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

In the generic descriptions of compounds of this invention, the number of atoms of a particular type in a substituent group is generally given as a range, e.g., an alkyl group containing from 1 to 4 carbon atoms or C₁₋₄ alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 12 ring carbon atoms, inclusive. Exemplary cyclic groups include cyclopropyl, cyclobutyl, cyclopentyl, and cyclohexyl groups.

By “C₁₋₄ alkyl” is meant a branched or unbranched hydrocarbon group having from 1 to 4 carbon atoms. A C₁₋₄ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₄ alkyls include, without limitation, methyl, ethyl, n-propyl, isopropyl, cyclopropyl, cyclopropylmethyl, n-butyl, iso-butyl, sec-butyl, tert-butyl, and cyclobutyl.

By “C₂₋₄ alkenyl” is meant a branched or unbranched hydrocarbon group containing one or more double bonds and having from 2 to 4 carbon atoms. A C₂₋₄ alkenyl may optionally include monocyclic or polycyclic rings, in which each ring desirably has from three to six members. The C₂₋₄ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₄ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl.

By “C₂₋₄ alkynyl” is meant a branched or unbranched hydrocarbon group containing one or more triple bonds and having from 2 to 4 carbon atoms. A C₂₋₄ alkynyl may optionally include monocyclic, bicyclic, or tricyclic rings, in which each ring desirably has five or six members. The C₂₋₄ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₄ alkynyls include, without limitation, ethynyl, 1-propynyl, 2-propynyl, 1-butynyl, 2-butynyl, and 3-butynyl.

By “C₂₋₆ heterocyclyl” is meant a stable 5- to 7-membered monocyclic or 7- to 14-membered bicyclic heterocyclic ring which is saturated, partially unsaturated, or unsaturated (aromatic), and which consists of 2 to 6 carbon atoms and 1, 2, 3, or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tettrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3, or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁₋₇ heteroalkyls include, without limitation, methoxymethyl and ethoxyethyl.

By “halide” or “halogen” is meant bromine, chlorine, iodine, or fluorine.

By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl, heteroalkyl, heteroaryl, and/or aryl groups, resulting in a positive charge at the nitrogen atom.

Other features and advantages of the invention will be apparent from the following Detailed Description, the drawings, and the claims.

### BRIEF DESCRIPTION OF THE DRAWINGS

FIG. 1 is a graph showing survival data for sertraline and oseltamivir in the lethal infection of influenza A/PR/8/34 induced in C57/BL6 mice.

FIG. 2 is a graph showing dose dependant increase in survival rate of sertraline-treated groups as compared to vehicle-treated groups.

### DETAILED DESCRIPTION

We have identified agents and combinations of agents that reduce the inflammatory response observed upon influenza infection in vitro and in vivo, and thus may be useful in treating or preventing viral infections, for example, those caused by an influenza virus. On this basis, the invention provides compositions, methods, and kits for treating a viral infection. The agents and combinations of agents described herein can reduce cytokine and chemokine expression and thus prevent or reduce symptoms associated with viral infections (e.g., influenza).

In particular, we have shown that the exemplary SSRI, sertraline, can be used to reduce the mortality associated with an influenza viral infection. On this basis, the invention features methods for treating or preventing viral infections, such as influenza, using an SSRI either alone or in combination with another agent (e.g., a corticosteroid, or an antiviral agent). The invention also features compositions including an SSRI and an antiviral agent, and kits including an SSRI (e.g., sertraline).

We have also identified the combinations of agents shown in Table 1 as being capable of reducing cytokine and chemokine expression in cells infected with an influenza virus. Because inflammation in the lung has been identified as playing an important role in mortality associated with influenza infection, one strategy to reduce the mortality rates associated with such infections is to reduce lung inflammation. In particular, these combinations of agents are observed to reduce expression of one or more of TNF-alpha, IFN-beta, IP-10, IL-6, IL-8, monocyte chemotactic protein-1 (MCP-1) and, in addition, can reduce influenza H₅N₁ matrix gene expression.

### Viruses

The invention relates to the treatment of viral disease, which can be caused by viruses from the families orthomyxoviridae, adenoviridae, paramyxoviridae, and coronaviridae. Virus of the orthomyxoviridae family include the influenza A virus, influenza B virus, influenza C virus, the infectious salmon anemia virus (isavirus), Thogoto Virus, and Dhori Virus. Members of the adenoviridae family include human adenovirus A, B, C, D, E, and F; bovine adenovirus A, B, and C; canine adenovirus; equine adenovirus A and B; murine adenovirus A; ovine adenovirus A and B; porcine adenovirus A, B, and C; and tree shrew adenovirus. Members of the paramyxoviridae family include bovine parainfluenza virus 3 (BPIV-3), human parainfluenza virus 1 (HPIV-1), human parainfluenza virus 3 (HPIV-3); sendai virus (murine parainfluenza virus 1); simian parainfluenza virus 10 (SPIV-10), bovine respiratory syncytial virus (BRSV), human respiratory syncytial virus (HRSV), pneumonia virus of mice (PVM), canine distemper virus (CDV), dolphin distemper virus (DMV), measles virus (MeV), Peste des petits ruminants virus (PPRV), phocine (seal) distemper virus (PDV), porpoise distemper virus, rinderpest virus (RPV), avian paramyxovirus 2 (APMV-2), avian paramyxovirus 3 (APMV-3), avian paramyxovirus 4 (APMV-4), avian paramyxovirus 5 (APMV-5), avian paramyxovirus 6 (APMV-6), avian paramyxovirus 7 (APMV-7), avian paramyxovirus 8 (APMV-8), avian paramyxovirus 9 (APMV-9), human parainfluenza virus 2 (HPIV-2), human parainfluenza virus 4a (HPIV-4a), human parainfluenza virus 4b (HPIV-4-b), mumps virus, newcastle disease virus (avian paramyxovirus 1) (NDV; APMV-1), porcine rubulavirus, simian parainfluenza virus 5 (SV-5), and simian parainfluenza virus 41 (SV-41). Members of the coronaviridae family include infectious bronchitis virus, bovine coronavirus, canine coronviarus, feline coronavirus, human coronavirus, and SARS-coronavirus.

Influenza Types, Subtypes, and Strains

In certain embodiments, the virus is an influenza virus. Influenza viruses are RNA viruses of the family Orthomyxoviridae. Three types of influenza viruses (types A, B, and C) have been identified. Subtypes of type A are based on variations in the hemagglutinin (HA) polypeptide and the neuraminidase (N) polypeptide. Fifteen (H1, H2, H3, H4, H5, H6, H7, H8, H9, H10, H11, H12, H13, H14, and H15) different HA subtypes have been identified, and nine (N1, N2, N3, N4, N5, N6, N7, N8, and N9) N subtypes have been identified. Strains including these subtypes can occur in various combinations (e.g., H1N1, H2N2, H3N2, H5N1, H7N7, H1N2, H9N2, H7N2, H7N3, H10N7). One serotype of infleunza B has been identified, and influenza type C is generally less virulent that types A or B.

Influenza Symptoms

Influenza is characterized by fever, headache, tiredness, cough, sore throat, runny or stuffy nose, body aches, and diarrhea and vomiting. Complications which can develop from an influenza infection include bacterial pneumonia, dehydration, and worsening of chronic medical conditions, such as congestive heart failure, asthma or diabetes. Sinus problems and ear infections can also develop.

Mortality due to influenza infection is often associated with lung inflammation, which can be severe. Influenza virus can induce cytokines including interleukin-6, interleukin-8, interleukin-10, and tumor necrosis factor-alpha in the serum and nasopharyngeal fluid (Laurent et. al., J Med Virol 64:262-268, 2001; Hayden et. al., J Clin Investig 101:643-649, 1998). Mortality associated with influenza infection is often due to the ability of the influenza A virus to infect the entire lung and induce high levels of macrophage-derived chemokines and cytokines, which results in infiltration of inflammatory cells and severe haemorrhage (Kobasa et. al., Nature 431:703-707, 2004).

### Compounds

Certain compounds that may be employed as agents in the methods, compositions, and kits of the present invention are discussed in greater detail below. It will be understood that analogs of any these compound can be used in the methods, compositions, and kits of the present invention. Additional information regarding these compounds can be found in U.S. Pat. Nos. 6,897,206 and 7,253,155 and U.S. Pat. Application Publication Nos. 2004/0220153, 2004/0224876, 2005/0192261, 2005/0119160, 2005/0187200, 2006/0286177, and 2005/0112199, each of which is incorporated by reference.

### Selective Serotonin Reuptake Inhibitors

The methods, compositions, and kits of the invention can include an SSRI or an analog thereof. Suitable SSRIs include cericlamine (e.g., cericlamine hydrochloride); citalopram (e.g., citalopram hydrobromide); clovoxamine; cyanodothiepin; dapoxetine; escitalopram (escitalopram oxalate); femoxetine (e.g., femoxetine hydrochloride); fluoxetine (e.g., fluoxetine hydrochloride); fluvoxamine (e.g., fluvoxamine maleate); ifoxetine; indalpine (e.g., indalpine hydrochloride); indeloxazine (e.g., indeloxazine hydrochloride); litoxetine; milnacipran (e.g., minlacipran hydrochloride); paroxetine (e.g., paroxetine hydrochloride hemihydrate; paroxetine maleate; paroxetine mesylate); sertraline (e.g., sertraline hydrochloride); tametraline hydrochloride; viqualine; and zimeldine (e.g., zimeldine hydrochloride).

Sertraline

Sertraline has the following structure:

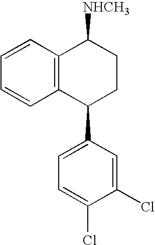

Structural analogs of sertraline are those having the formula:

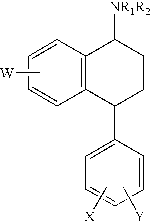

wherein R₁ is selected from the group consisting of hydrogen and C₁₋₄ alkyl; R₂ is C₁₋₄ alkyl; X and Y are each selected from the group consisting of hydrogen, fluoro, chloro, bromo, trifluoromethyl, C₁₋₃ alkoxy, and cyano; and W is selected from the group consisting of hydrogen, fluoro, chloro, bromo, trifluoromethyl and C₁₋₃ alkoxy. Preferred sertraline analogs are in the cis-isomeric configuration. The term “cis-isomeric” refers to the relative orientation of the NR₁R₂ and phenyl moieties on the cyclohexene ring (i.e. they are both oriented on the same side of the ring). Because both the 1- and 4-carbons are asymmetrically substituted, each cis-compound has two optically active enantiomeric forms denoted (with reference to the 1-carbon) as the cis-(1R) and cis-(1S) enantiomers. Sertraline analogs are also described in U.S. Pat. No. 4,536,518. Other related compounds include (S,S)—N-desmethylsertraline, rac-cis-N-desmethylsertraline, (1S,4S)-desmethyl sertraline, 1-des (methylamine)-1-oxo-2-(R,S)-hydroxy sertraline, (1R,4R)-desmethyl sertraline, sertraline sulfonamide, sertraline (reverse) methanesulfonamide, 1R,4R sertraline enantiomer, N,N-dimethyl sertraline, nitro sertraline, sertraline aniline, sertraline iodide, sertraline sulfonamide NH₂, sertraline sulfonamide ethanol, sertraline nitrile, sertraline-CME, dimethyl sertraline reverse sulfonamide, sertraline reverse sulfonamide (CH₂ linker), sertraline B-ring ortho methoxy, sertraline A-ring methyl ester, sertraline A-ring ethanol, sertraline N,N-dimethylsulfonamide, sertraline A ring carboxylic acid, sertraline B-ring para-phenoxy, sertraline B-ring para-trifluoromethane, N,N-dimethyl sertraline B-Ring para-trifluoromethane, and UK-416244. Structures of these analogs are shown below.

| Name | Structure |
|---|---|
| (1R,4S) Sertraline Hydrochloride | 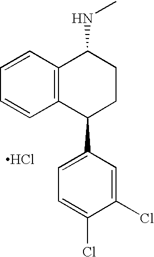 |
| (1S,4R) Sertraline Hydrochloride | 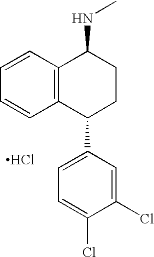 |
| Sertraline B-Ring Para-Phenoxy | 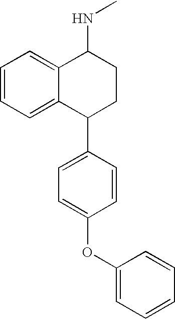 |
| Sertraline B-Ring Ortho-Methoxy | 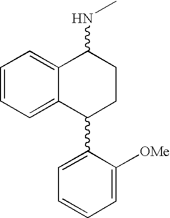 |
| 1R,4R Sertraline Enantiomer | 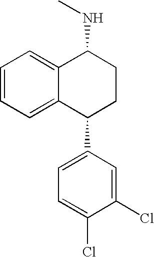 |
| Sertraline Sulfonamide | 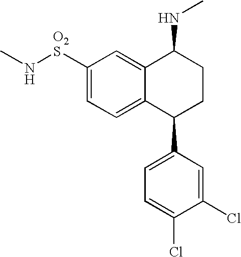 |
| Nitro Sertraline | 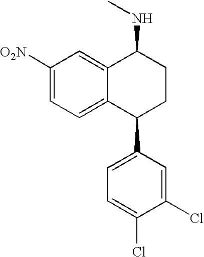 |
| Sertraline Aniline | 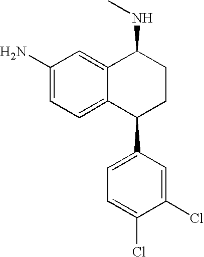 |
| Sertraline Reverse Sulfonamide (CH2 linker) | 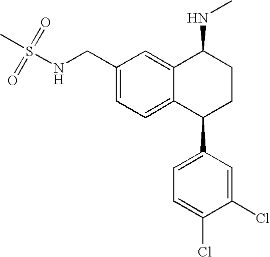 |
| UK-416244 | 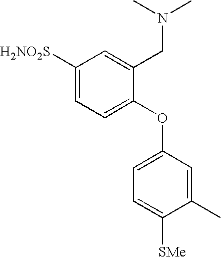 |
| (1R,4R)-Desmethyl Sertraline | 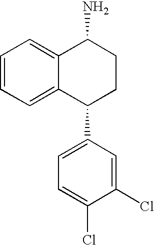 |
| Sertraline A-Ring Methyl Ester | 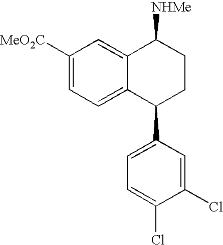 |
| rac-cis-N-Desmethyl Sertraline, Hydrochloride | 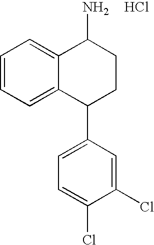 |
| Dimethyl Sertraline Reverse Sulfonamide | 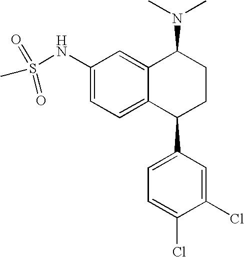 |
| Sertraline N,N- Dimethylsulfonamide | 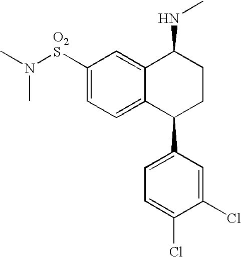 |
| Sertraline A-Ring Ethanol | 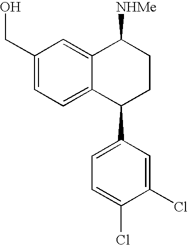 |
| Sertraline-CME | 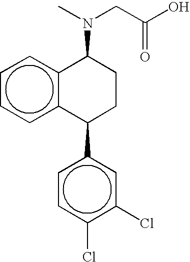 |
| (1S,4S)-Desmethyl Sertraline, Hydrochloride | 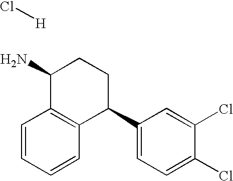 |
| Sertraline Iodide | 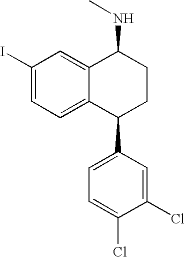 |
| 1-Des(methylamine)- 1-oxo-2-(R,S)- hydroxy Sertraline | 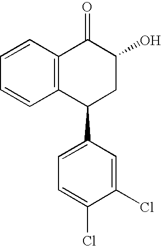 |
| Sertraline Nitrile | 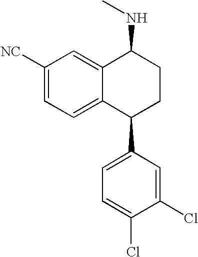 |
| Sertraline Hydrochloride | 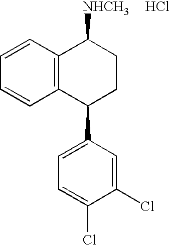 |
| N,N-Dimethyl Sertraline B-Ring Para- Trifluoromethane | 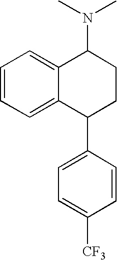 |
| Sertraline Sulfonamide NH2 | 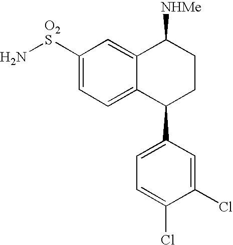 |
| Sertraline (Reverse) Methanesulfonamide | 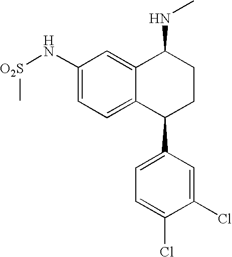 |
| Sertraline A-Ring Carboxylic Acid | 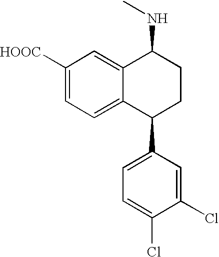 |
| Sertraline Sulfonamide Ethanol | 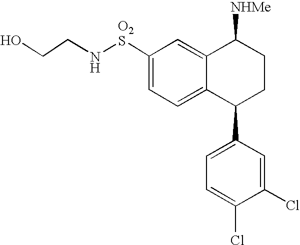 |
| Sertraline B-Ring Para-Trifluoromethane | 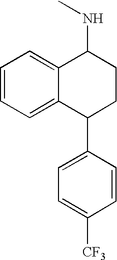 |
| N,N-Dimethyl Sertraline | 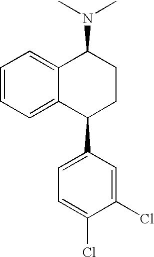 |

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

Cericlamine

Cericlamine has the following structure:

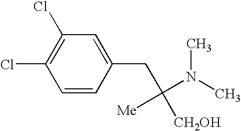

Structural analogs of cericlamine are those having the formula:

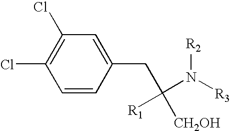

as well as pharmaceutically acceptable salts thereof, wherein R₁ is a C₁-C₄ alkyl and R₂ is H or C₁₋₄ alkyl, R₃ is H, C₁₋₄ alkyl, C₂₋₄ alkenyl, phenylalkyl or cycloalkylalkyl with 3 to 6 cyclic carbon atoms, alkanoyl, phenylalkanoyl or cycloalkylcarbonyl having 3 to 6 cyclic carbon atoms, or R₂ and R₃ form, together with the nitrogen atom to which they are linked, a heterocycle saturated with 5 to 7 chain links which can have, as the second heteroatom not directly connected to the nitrogen atom, an oxygen, a sulphur or a nitrogen, the latter nitrogen heteroatom possibly carrying a C₂₋₄ alkyl.

Exemplary cericlamine structural analogs are 2-methyl-2-amino-3-(3,4-dichlorophenyl)-propanol, 2-pentyl-2-amino-3-(3,4-dichlorophenyl)-propanol, 2-methyl-2-methylamino-3-(3,4-dichlorophenyl)-propanol, 2-methyl-2-dimethylamino-3-(3,4-dichlorophenyl)-propanol, and pharmaceutically acceptable salts of any thereof.

Citalopram

Citalopram has the following structure:

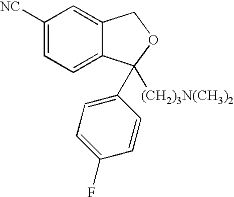

Structural analogs of citalopram are those having the formula:

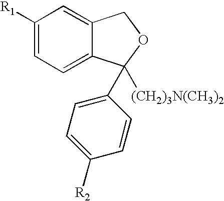

as well as pharmaceutically acceptable salts thereof, wherein each of R₁ and R₂ is independently selected from the group consisting of bromo, chloro, fluoro, trifluoromethyl, cyano and R—CO—, wherein R is C₁₋₄ alkyl.

Exemplary citalopram structural analogs (which are thus SSRI structural analogs according to the invention) are 1-(4′-fluorophenyl)-1-(3-dimethylaminopropyl)-5-bromophthalane; 1-(4′-chlorophenyl)-1-(3-dimethylaminopropyl)-5-chlorophthalane; 1-(4′-bromophenyl)-1-(3-dimethylaminopropyl)-5-chlorophthalane; 1-(4′-fluorophenyl)-1-(3-dimethylaminopropyl)-5-chlorophthalane; 1-(4′-chlorophenyl)-1-(3-dimethylaminopropyl)-5-trifluoromethyl-phthalane; 1-(4′-bromophenyl)-1-(3-dimethylaminopropyl)-5-trifluoromethyl-phthalane; 1-(4′-fluorophenyl)-1-(3-dimethylaminopropyl)-5-trifluoromethyl-phthalane; 1-(4′-fluorophenyl)-1-(3-dimethylaminopropyl)-5-fluorophthalane; 1-(4′-chlorophenyl)-1-(3-dimethylaminopropyl)-5-fluorophthalane; 1-(4′-chlorophenyl)-1-(3-dimethylaminopropyl)-5-phthalancarbonitrile; 1-(4′-fluorophenyl)-1-(3-dimethylaminopropyl)-5-phthalancarbonitrile; 1-(4′-cyanophenyl)-1-(3-dimethylaminopropyl)-5-phthalancarbonitrile; 1-(4′-cyanophenyl)-1-(3-dimethylaminopropyl)-5-chlorophthalane; 1-(4′-cyanophenyl)-1-(3-dimethylaminopropyl)-5-trifluoromethylphthalane; 1-(4′-fluorophenyl)-1-(3-dimethylaminopropyl)-5-phthalancarbonitrile; 1-(4′-chlorophenyl)-1-(3-dimethylaminopropyl)-5-ionylphthalane; 1-(4-(chlorophenyl)-1-(3-dimethylaminopropyl)-5-propionylphthalane; and pharmaceutically acceptable salts of any thereof. Additional analogs are described in U.S. Pat. No. 4,136,193.

Clovoxamine

Clovoxamine has the following structure:

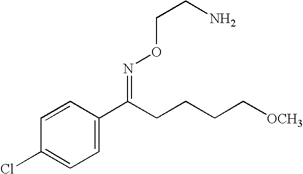

Structural analogs of clovoxamine are those having the formula:

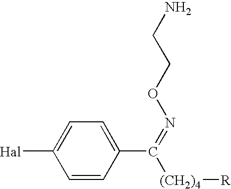

as well as pharmaceutically acceptable salts thereof, wherein Hal is a chloro, bromo, or fluoro group and R is a cyano, methoxy, ethoxy, methoxymethyl, ethoxymethyl, methoxyethoxy, or cyanomethyl group.

Exemplary clovoxamine structural analogs are 4′-chloro-5-ethoxyvalerophenone O-(2-aminoethyl)oxime; 4′-chloro-5-(2-methoxyethoxy)valerophenone O-(2-aminoethyl)oxime; 4′-chloro-6-methoxycaprophenone O-(2-aminoethyl)oxime; 4′-chloro-6-ethoxycaprophenone O-(2-aminoethyl)oxime; 4′-bromo-5-(2-methoxyethoxy)valerophenone O-(2-aminoethyl)oxime; 4′-bromo-5-methoxyvalerophenone O-(2-aminoethyl)oxime; 4′-chloro-6-cyanocaprophenone O-(2-aminoethyl)oxime; 4′-chloro-5-cyanovalerophenone O-(2-aminoethyl)oxime; 4′-bromo-5-cyanovalerophenone O-(2-aminoethyl)oxime; and pharmaceutically acceptable salts of any thereof.

Femoxetine

Femoxetine has the following structure:

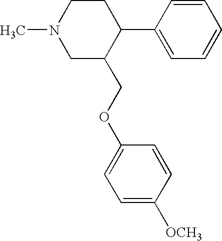

Structural analogs of femoxetine are those having the formula:

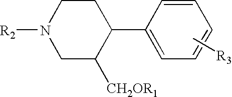

wherein R₁ represents a C₁₋₄ alkyl or C₂₋₄ alkynyl group, or a phenyl group optionally substituted by C₁₋₄ alkyl, C₁₋₄ alkylthio, C₁₋₄ alkoxy, bromo, chloro, fluoro, nitro, acylamino, methylsulfonyl, methylenedioxy, or tetrahydronaphthyl, R₂ represents a C₁₋₄ alkyl or C₂₋₄ alkynyl group, and R₃ represents hydrogen, C₁₋₄ alkyl, C₁₋₄alkoxy, trifluoroalkyl, hydroxy, bromo, chloro, fluoro, methylthio, or aralkyloxy.

Exemplary femoxetine structural analogs are disclosed in Examples 7-67 of U.S. Pat. No. 3,912,743, hereby incorporated by reference.

Fluoxetine

Fluoxetine has the following structure:

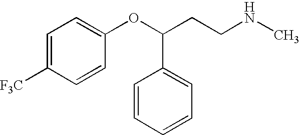

Structural analogs of fluoxetine are those compounds having the formula:

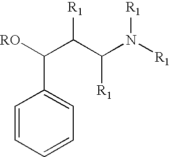

as well as pharmaceutically acceptable salts thereof, wherein each R₁ is independently hydrogen or methyl; R is naphthyl or

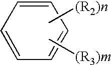

wherein each of R₂ and R₃ is, independently, bromo, chloro, fluoro, trifluoromethyl, C₁₋₄ alkyl, C₁₋₃ alkoxy or C₃₋₄ alkenyl; and each of n and m is, independently, 0, 1 or 2. When R is naphthyl, it can be either α-naphthyl or 3-naphthyl.

Exemplary fluoxetine structural analogs are 3-(p-isopropoxyphenoxy)-3-phenylpropylamine methanesulfonate, N,N-dimethyl 3-(3′,4′-dimethoxyphenoxy)-3-phenylpropylamine p-hydroxybenzoate, N,N-dimethyl 3-(α-naphthoxy)-3-phenylpropylamine bromide, N,N-dimethyl 3-(β-naphthoxy)-3-phenyl-1-methylpropylamine iodide, 3-(2′-methyl-4′,5′-dichlorophenoxy)-3-phenylpropylamine nitrate, 3-(p-t-butylphenoxy)-3-phenylpropylamine glutarate, N-methyl 3-(2′-chloro-p-tolyloxy)-3-phenyl-1-methylpropylamine lactate, 3-(2′,4′-dichlorophenoxy)-3-phenyl-2-methylpropylamine citrate, N,N-dimethyl 3-(m-anisyloxy)-3-phenyl-1-methylpropylamine maleate, N-methyl 3-(p-tolyloxy)-3-phenylpropylamine sulfate, N,N-dimethyl 3-(2′,4′-difluorophenoxy)-3-phenylpropylamine 2,4-dinitrobenzoate, 3-(o-ethylphenoxy)-3-phenylpropylamine dihydrogen phosphate, N-methyl 3-(2′-chloro-4′-isopropylphenoxy)-3-phenyl-2-methylpropylamine maleate, N,N-dimethyl 3-(2′-alkyl-4′-fluorophenoxy)-3-phenyl-propylamine succinate, N,N-dimethyl 3-(o-isopropoxyphenoxy)-3-phenyl-propylamine phenylacetate, N,N-dimethyl 3-(o-bromophenoxy)-3-phenyl-propylamine β-phenylpropionate, N-methyl 3-(p-iodophenoxy)-3-phenyl-propylamine propiolate, and N-methyl 3-(3-n-propylphenoxy)-3-phenyl-propylamine decanoate. Additional flouxetine analogs are described in U.S. Pat. No. 4,314,081.

Fluvoxamine

Fluvoxamine has the following structure:

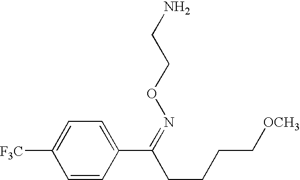

Structural analogs of fluvoxamine are those having the formula:

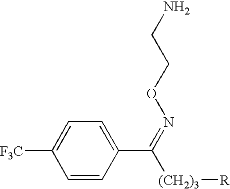

as well as pharmaceutically acceptable salts thereof, wherein R is cyano, cyanomethyl, methoxymethyl, or ethoxymethyl. Analogs of fluvoxamine are also described in U.S. Pat. No. 4,085,225.

Indalpine

Indalpine has the following structure:

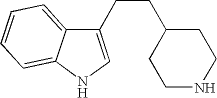

Structural analogs of indalpine are those having the formula:

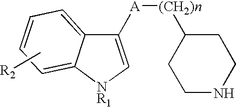

or pharmaceutically acceptable salts thereof, wherein R₁ is a hydrogen atom, a C₁-C₄ alkyl group, or an aralkyl group of which the alkyl has 1 or 2 carbon atoms, R₂ is hydrogen, C₁₋₄ alkyl, C₁₋₄ alkoxy or C₁₋₄ alkylthio, chloro, bromo, fluoro, trifluoromethyl, nitro, hydroxy, or amino, the latter optionally substituted by one or two C₁₋₄ alkyl groups, an acyl group or a C₁₋₄alkylsulfonyl group; A represents —CO or —CH₂— group; and n is 0, 1 or 2.

Exemplary indalpine structural analogs are indolyl-3 (piperidyl-4 methyl) ketone; (methoxy-5-indolyl-3) (piperidyl-4 methyl) ketone; (chloro-5-indolyl-3) (piperidyl-4 methyl) ketone; (indolyl-3)-1(piperidyl-4)-3 propanone, indolyl-3 piperidyl-4 ketone; (methyl-1 indolyl-3) (piperidyl-4 methyl) ketone, (benzyl-1 indolyl-3) (piperidyl-4 methyl) ketone; [(methoxy-5 indolyl-3)-2 ethyl]-piperidine, [(methyl-1 indolyl-3)-2 ethyl]-4-piperidine; [(indolyl-3)-2 ethyl]-4 piperidine; (indolyl-3 methyl)-4 piperidine, [(chloro-5 indolyl-3)-2 ethyl]-4 piperidine; [(indolyl-b 3)-3 propyl]-4 piperidine; [(benzyl-1 indolyl-3)-2 ethyl]-4 piperidine; and pharmaceutically acceptable salts of any thereof. Additional indalpine derivatives are described in U.S. Pat. No. 4,064,255.

Indeloxazine

Indeloxezine has the following structure:

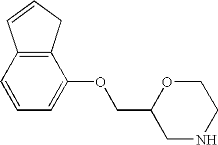

Structural analogs of indeloxazine are those having the formula:

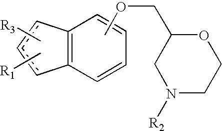

and pharmaceutically acceptable salts thereof, wherein R₁ and R₃ each represents hydrogen, C₁₋₄ alkyl, or phenyl; R₂ represents hydrogen, C₁₋₄ alkyl, C₄₋₇ cycloalkyl, phenyl, or benzyl; one of the dotted lines means a single bond and the other means a double bond, or the tautomeric mixtures thereof.

Exemplary indeloxazine structural analogs are 2-(7-indenyloxymethyl)-4-isopropylmorpholine; 4-butyl-2-(7-indenyloxymethyl)morpholine; 2-(7-indenyloxymethyl)-4-methylmorpholine; 4-ethyl-2-(7-indenyloxymethyl)morpholine, 2-(7-indenyloxymethyl)-morpholine; 2-(7-indenyloxymethyl)-4-propylmorpholine; 4-cyclohexyl-2-(7-indenyloxymethyl)morpholine; 4-benzyl-2-(7-indenyloxymethyl)-morpholine; 2-(7-indenyloxymethyl)-4-phenylmorpholine; 2-(4-indenyloxymethyl)morpholine; 2-(3-methyl-7-indenyloxymethyl)-morpholine; 4-isopropyl-2-(3-methyl-7-indenyloxymethyl)morpholine; 4-isopropyl-2-(3-methyl-4-indenyloxymethyl)morpholine; 4-isopropyl-2-(3-methyl-5-indenyloxymethyl)morpholine; 4-isopropyl-2-(1-methyl-3-phenyl-6-indenyloxymethyl)morpholine; 245-indenyloxymethyl)-4-isopropyl-morpholine, 2-(6-indenyloxymethyl)-4-isopropylmorpholine; and 4-isopropyl-2-(3-phenyl-6-indenyloxymethyl)morpholine; as well as pharmaceutically acceptable salts of any thereof. Additional Indeloxazine analogs are described in U.S. Pat. No. 4,109,088

Milnacipran

Milnacipran has the following structure:

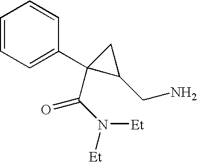

Structural analogs of milnacipran are those having the formula:

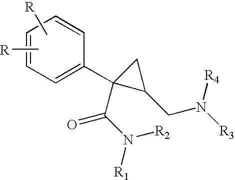

as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipran structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof. Additional milnaciprain analogs are described in U.S. Pat. No. 4,478,836.

Paroxetine

Paroxetine has the following structure:

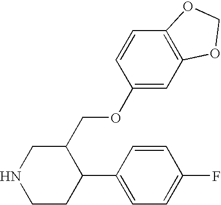

Structural analogs of paroxetine are those having the formula:

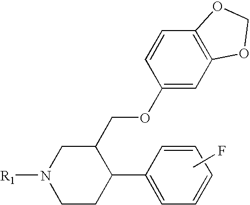

and pharmaceutically acceptable salts thereof, wherein R₁ represents hydrogen or a C₁₋₄ alkyl group, and the fluorine atom may be in any of the available positions.

Zimeldine

Zimeldine has the following structure:

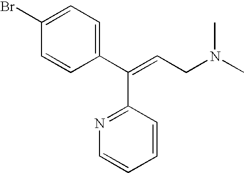

Structural analogs of zimeldine are those compounds having the formula:

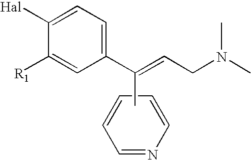

and pharmaceutically acceptable salts thereof, wherein the pyridine nucleus is bound in ortho-, meta- or para-position to the adjacent carbon atom and where R₁ is selected from the group consisting of H, chloro, fluoro, and bromo.

Exemplary zimeldine analogs are (e)- and (z)-3-(4′-bromophenyl-3-(2″-pyridyl)-dimethylallylamine; 3-(4′-bromophenyl)-3-(3″-pyridyl)-dimethylallylamine; 3-(4′-bromophenyl)-3-(4″-pyridyl)-dimethylallylamine; and pharmaceutically acceptable salts of any thereof. Zimelidine analogs are also described in U.S. Pat. No. 3,928,369.

Structural analogs of any of the above SSRIs are considered herein to be SSRI analogs and thus may be employed in any of the methods, compositions, and kits of the invention.

### Metabolites

Pharmacologically active metabolites of any of the foregoing SSRIs can also be used in the methods, compositions, and kits of the invention. Exemplary metabolites are didesmethylcitalopram, desmethylcitalopram, desmethylsertraline, and norfluoxetine.

### Analogs

Functional analogs of SSRIs can also be used in the methods, compositions, and kits of the invention. Exemplary SSRI functional analogs are provided below. One class of SSRI analogs includes SNRIs (selective serotonin norepinephrine reuptake inhibitors), which include venlafaxine, duloxetine, and 4-(2-fluorophenyl)-6-methyl-2-piperazinothieno[2,3-d]pyrimidine.

Venlafaxine

Venlafaxine hydrochloride (EFFEXOR™) is an antidepressant for oral administration. It is designated (R/S)-1-[2-(dimethylamino)-1-(4-methoxyphenyl)ethyl]cyclohexanol hydrochloride or (±)-1-[(alpha)-[(dimethyl-amino)methyl]-p-methoxybenzyl]cyclohexanol hydrochloride. Compressed tablets contain venlafaxine hydrochloride equivalent to 25 mg, 37.5 mg, 50 mg, 75 mg, or 100 mg venlafaxine. The recommended starting dose for venlafaxine is 75 mg/day, administered in two or three divided doses, taken with food. Depending on tolerability and the need for further clinical effect, the dose may be increased to 150 mg/day. If desirable, the dose can be further increased up to 225 mg/day. When increasing the dose, increments of up to 75 mg/day are typically made at intervals of no less than four days.

Venlafaxine has the following structure:

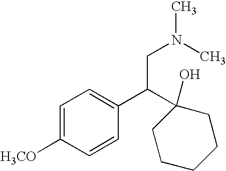

Structural analogs of venlafaxine are those compounds having the formula:

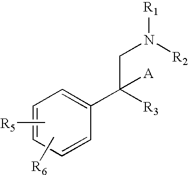

as well as pharmaceutically acceptable salts thereof, wherein A is a moiety of the formula:

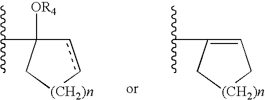

where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.

Duloxetine

Duloxetine has the following structure:

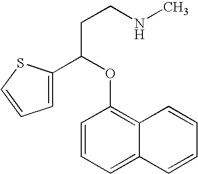

Structural analogs of duloxetine are those compounds described by the formula disclosed in U.S. Pat. No. 4,956,388, hereby incorporated by reference.

Other SSRI analogs are 4-(2-fluorophenyl)-6-methyl-2-piperazinothieno[2,3-d]pyrimidine, 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; 0-desmethylvenlafaxine; WY 45,818; WY 45,881; N-(3-fluoropropyl)paroxetine; Lu 19005; and SNRIs described in PCT Publication No. WO 04/004734.

### SSRI Standard Recommended Dosages

Standard recommended dosages for exemplary SSRIs are provided in Table 4, below. Other standard dosages are provided, e.g., in the Merck Manual of Diagnosis & Therapy (17th Ed. MH Beers et al., Merck & Co.) and Physicians' Desk Reference 2003 (57^{th} Ed. Medical Economics Staff et al., Medical Economics Co., 2002).

TABLE 4

| Compound | Standard Dose | |
|---|---|---|
| Fluoxetine | 20-80 | mg/day |
| Sertraline | 50-200 | mg/day |
| Paroxetine | 20-50 | mg/day |
| Fluvoxamine | 50-300 | mg/day |
| Citalopram | 10-80 | mg qid |
| Escitalopram | 10 | mg qid |

In some embodiments of the invention, it may be desirable to administer SSRIs at doses higher or lower than a standard dosing. For example, sertraline may be administered at doses 100%, 200%, 500%, or 1000% fold greater than a dose falling with the range of the standard doses.

### Corticosteroids

In certain embodiments, a corticosteroid can be used in the compositions, methods, and kits of the invention. Corticosteroids include prednisolone, budesonide, and any of those described herein.

Prednisolone

Prednisolone has the following structure:

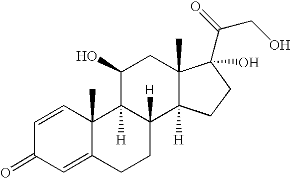

Analogs of prednisolone are described in U.S. Pat. No. 3,134,718 and have the formula:

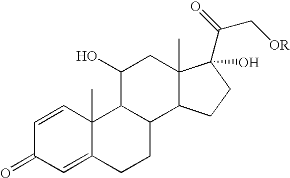

where R is selected from the group of hydrogen and lower alkanoyl.

Budesonide

Budesonide has the following structure:

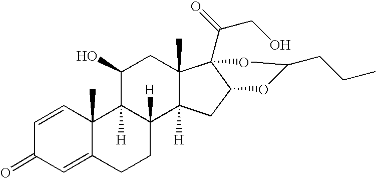

Structural analogs of budesonide are those compounds having the formula:

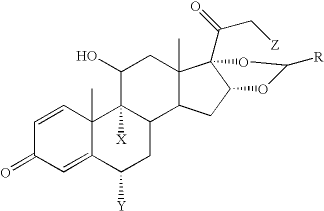

where X and Y are independently selected from hydrogen and fluorine, X being selected from hydrogen and fluorine when Y is hydrogen and X being fluorine when Y is fluorine, Z is selected from hydroxyl and esterified hydroxyl preferably containing a maximum of 12 carbon atoms, if any, in the esterifying group, R is selected from straight and branched hydrocarbon chains having 2-10 and preferably 2-6 carbon atoms. Analogs are described in U.S. Pat. No. 3,929,768.

Other Corticosteroids

Other corticosteroids that may be used in the compositions, methods, and kits of the invention include 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta,21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone (analogs described in U.S. Pat. No. 4,472,393); mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone 21-diethylaminoacetate; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin or derivatives thereof (see, e.g., U.S. Pat. No. 7,081,475).

Steroid Receptor Modulators

Steroid receptor modulators (e.g., antagonists and agonists) may be used as a substitute for or in addition to a corticosteroid in the compositions, methods, and kits of the invention.

Glucocorticoid receptor modulators that may used in the compositions, methods, and kits of the invention include compounds described in U.S. Pat. Nos. 6,380,207, 6,380,223, 6,448,405, 6,506,766, and 6,570,020, U.S. Pat. Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO 00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

### Tricyclic Antidepressants

Tricyclic antidepressants include compounds having one of the formulas (I), (II), (III), or (IV):

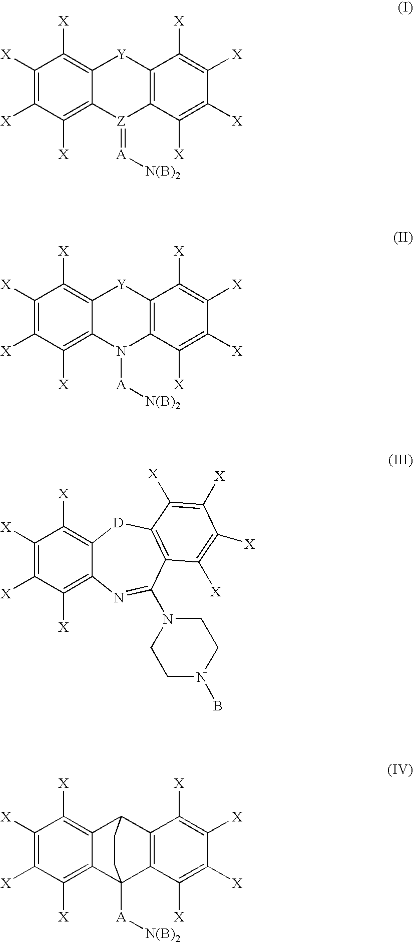

wherein each X is, independently, H, Cl, F, Br, I, CH₃, CF₃, OH, OCH₃, CH₂CH₃, or OCH₂CH₃; Y is CH₂, O, NH, S(O)₀₋₂, (CH₂)₃, (CH)₂, CH₂O, CH₂NH, CHN, or CH₂S; Z is C or S; A is a branched or unbranched, saturated or mono-unsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, S(O)₀₋₂.

In preferred embodiments, each X is, independently, H, Cl, or F; Y is (CH₂)₂, Z is C; A is (CH₂)₃; and each B is, independently, H, Cl, or F.

Exemplary tricyclic antidepressants are maprotiline, amoxapine, 8-hydroxyamoxapine, 7-hydroxyamoxapine, loxapine, loxapine succinate, loxapine hydrochloride, 8-hydroxyloxapine, amitriptyline, clomipramine, doxepin, imipramine, trimipramine, desipramine, nortriptyline, and protriptyline.

Amoxapine

In certain embodiments, amoxapine or an amoxapine analog can be used in the compositions, methods, and kits of the invention. Amoxapine has the structure:

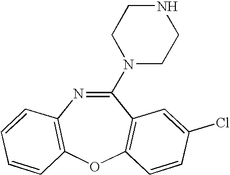

Amoxapine analogs include 8-hydroxyamoxapine, 7-hydroxyamoxapine, loxapine, loxapine succinate, loxapine hydrochloride, 8-hydroxyloxapine, clothiapine, perlapine, fluperlapine, and dibenz(b,f)(1,4)oxazepine, 2-chloro-11-(4-methyl-1-piperazinyl)-, monohydrochloride, N-acetylamoxapine, N-formyl-7-hydroxyamoxapine, and carboxymethyl ester-amoxapine (CME-amoxapine). CME-amoxapine has the structure:

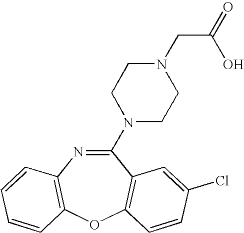

Analogs of CME-amoxapine are described in U.S. Pat. No. 5,344,828.

Nortriptyline

In certain embodiments, nortriptyline or a nortriptyline analog can be used in the compositions, methods, and kits of the invention. Nortriptyline has the structure:

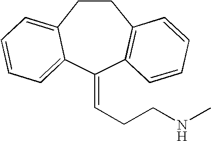

Nortriptyline analogs are described in U.S. Pat. No. 3,922,305 and have the structures:

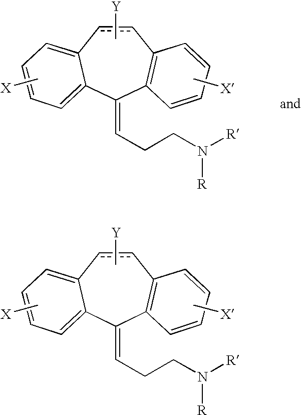

where R′ is hydrogen or a cyano radical; R is hydrogen or an alkyl or alkenyl radical having up to 6 carbons, either straight or branched chain, or cycloalkyl having up to 8 carbons or aralkyl groups such as benzyl; Y is hydrogen or halogen, preferably bromine or chlorine; X and X′ are similar or dissimilar and are selected from hydrogen, an alkyl group having up to 6 carbon atoms, an alkenyl group having up to 6 carbon atoms, a perfluoroalkyl group having up to 4 carbon atoms, a phenyl or a substituted phenyl radical, an acyl group having up to 4 carbon atoms, a perfluoroacyl group having up to 4 carbon atoms, amino, an alkylamino group having up to 4 carbon atoms, a dialkylamino group having up to 8 carbon atoms, an acylamino group having up to 4 carbon atoms, a perfluoroacylamino group having up to 4 carbon atoms, an alkylsulfonylamino group having up to 4 carbon atoms, halogen (fluorine, chlorine, bromine or iodine), hydroxyl, an alkoxyl group having up to 4 carbon atoms, a perfluoroalkoxyl group having up to 4 carbon atoms, cyano, carboxy, carbamoyl, an alkylcarbamoyl group having up to 5 carbon atoms, a dialkylcarbamoyl group having up to 9 carbon atoms, a carbalkoxy group having up to 6 carbon atoms, mercapto, an alkylmercapto group having up to 4 carbon atoms, a perfluoroalkylmercapto group having up to 4 carbon atoms, an alkylsulfonyl group having up to 4 carbon atoms, a perfluoroalkylsulfonyl group having up to 4 carbon atoms, sulfamoyl, an alkylsulfamoyl group having up to 4 carbon atoms, or a dialkylsulfamoyl group having up to 8 carbon atoms. More than one of these substituents may be on each benzenoid ring. The compounds may have substituents on the propyl or propylidene chain such as lower alkyl radicals, preferably having from 1 to 4 carbon atoms.

Nortriptyline analogs include 10-hydroxynortriptyline, 10-oxonortriptyline, desmethylnortriptyline, and fluphenazine.

### Bufexamac

In certain embodiments, bufexamac or a bufexamac analog can be used in the compositions, methods, and kits of the invention. By “bufexamac analog” is meant a compound having the formula (VI):

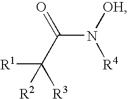

wherein R¹ is

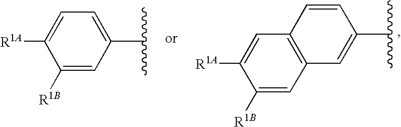

wherein R^{1A} is and R^{1B} is H, halo, CF₃, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₁₋₆ alkoxy, or optionally substituted C₁₋₆ thioalkoxy; each of R² and R³ is, independently, H, C₁₋₄ alkyl, or CF₃; and R⁴ is optionally substituted C₁₋₆ alkyl or optionally substituted C₃₋₈ cycloalkyl.

### Tetra-Substituted Pyrimidopyrimidines

In certain embodiments, a tetra-substituted pyrimidopyrimidine can be used in the compositions, methods, and kits of the invention.

Tetra-substituted pyrimidopyrimidines have the formula (V):

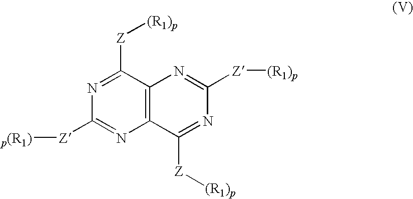

wherein each Z and each Z′ is, independently, N, O, C,

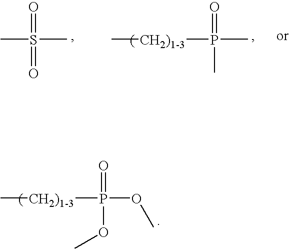

When Z or Z′ is O or

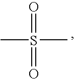

then p=1, when Z or Z′ is N,

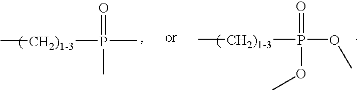

then p=2, and when Z or Z′ is C, then p=3. In formula (V), each R₁ is, independently, X, OH, N-alkyl (wherein the alkyl group has 1 to 20, more preferably 1-5, carbon atoms); a branched or unbranched alkyl group having 1 to 20, more preferably 1-5, carbon atoms; or a heterocycle, preferably as defined in formula (Y), below. Alternatively, when p>1, two R₁ groups from a common Z or Z′ atom, in combination with each other, may represent —(CY₂)ₖ— in which k is an integer between 4 and 6, inclusive. Each X is, independently, Y, CY₃, C(CY₃)₃, CY₂CY₃, (CY₂)₁₋₅OY, substituted or unsubstituted cycloalkane of the structure CₙY₂ₙ₋₁, wherein n=3-7, inclusive. Each Y is, independently, H, F, Cl, Br, or I. In one embodiment, each Z is the same moiety, each Z′ is the same moiety, and Z and Z′ are different moieties.

Tetra-substituted pyrimidopyrimidines that are useful in the methods, compositions, and kits of this invention include 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine); mopidamole; dipyridamole monoacetate; NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. Nos. 3,031,450 and 4,963,541. The standard recommended dosage for dipyridamole is 300-400 mg/day.

### Ibudilast

In certain embodiments, ibudilast or an ibudilast analog, as defined by formula below, may be used in the compositions, methods, and kits of the invention.

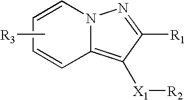

In this formula, R₁ and R₂ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; R₃ is selected from H, halide, alkoxy, and C₁₋₄ alkyl; X₁ is selected from C═O, C═N—NH—R₄, C═C(R₅)—C(O)—R₆, C═CH═CH—C(O)—R₆, and C(OH)—R₇; R₄ is selected from H and acyl; R₅ is selected from H, halide, and C₁₋₄ alkyl; R₆ is selected from OH, alkoxy and amido; and R₇ is selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl.

Compounds of formula (VI) include, the compounds described in U.S. Pat. Nos. 3,850,941; 4,097,483; 4,578,392; 4,925,849; 4,994,453; and 5,296,490. Commercially available compounds of formula (VI) include ibudilast and KC-764.

The standard recommended dosage for the treatment of bronchial asthma is typically 10 mg of ibudilast twice daily, while in the case of cerebrovascular disorders, the standard recommended dosage is 10 mg of ibudilast three times daily. The structure of ibudilast is shown below:

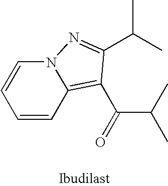

KC-764 (CAS 94457-09-7) is reported to be a platelet aggregation inhibitor. The structure of KC-764 is shown below:

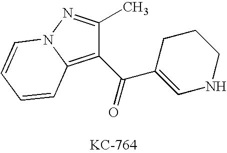

KC-764 and other compounds of formula (VI) can be prepared using the synthetic methods described in U.S. Pat. Nos. 3,850,941; 4,097,483; 4,578,392; 4,925,849; 4,994,453; and 5,296,490.

### Antihistamines

In certain embodiments, an antihistamine or an antihistamine analog can be used in the compositions, methods, and kits of the invention. Antihistamines are compounds that block the action of histamine. Classes of antihistamines include:

(1) Ethanolamines (e.g., bromodiphenhydramine, carbinoxamine, clemastine, dimenhydrinate, diphenhydramine, diphenylpyraline, and doxylamine);

(2) Ethylenediamines (e.g., pheniramine, pyrilamine, tripelennamine, and triprolidine);

(3) Phenothiazines (e.g., diethazine, ethopropazine, methdilazine, promethazine, thiethylperazine, and trimeprazine);

(4) Alkylamines (e.g., acrivastine, brompheniramine, chlorpheniramine, desbrompheniramine, dexchlorpheniramine, pyrrobutamine, and triprolidine);

(5) piperazines (e.g., buclizine, cetirizine, chlorcyclizine, cyclizine, meclizine, hydroxyzine);

(6) Piperidines (e.g., astemizole, azatadine, cyproheptadine, desloratadine, fexofenadine, loratadine, ketotifen, olopatadine, phenindamine, and terfenadine);

(7) Atypical antihistamines (e.g., azelastine, levocabastine, methapyrilene, and phenyltoxamine).

In the compositions, methods, and kits of the invention, both non-sedating and sedating antihistamines may be employed. Non-sedating antihistamines include loratadine and desloratadine. Sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other antihistamines suitable for use in the compositions, methods, and kits of the invention are acrivastine; ahistan; antazoline; astemizole; azelastine (e.g., azelsatine hydrochloride); bamipine; bepotastine; benztropine, bietanautine; brompheniramine (e.g., brompheniramine maleate); carbinoxamine (e.g., carbinoxamine maleate); cetirizine (e.g., cetirizine hydrochloride); cetoxime; chlorocyclizine; chloropyramine; chlorothen; chlorphenoxamine; cinnarizine; clemastine (e.g., clemastine fumarate); clobenzepam; clobenztropine; clocinizine; cyclizine (e.g., cyclizine hydrochloride; cyclizine lactate); deptropine; dexchlorpheniramine; dexchlorpheniramine maleate; diphenylpyraline; doxepin; ebastine; embramine; emedastine (e.g., emedastine difumarate); epinastine; etymemazine hydrochloride; fexofenadine (e.g., fexofenadine hydrochloride); histapyrrodine; hydroxyzine (e.g., hydroxyzine hydrochloride; hydroxyzine pamoate); isopromethazine; isothipendyl; levocabastine (e.g., levocabastine hydrochloride); mebhydroline; mequitazine; methafurylene; methapyrilene; metron; mizolastine; olapatadine (e.g., olopatadine hydrochloride); orphenadrine; phenindamine (e.g., phenindamine tartrate); pheniramine; phenyltoloxamine; p-methyldiphenhydramine; pyrrobutamine; setastine; talastine; terfenadine; thenyldiamine; thiazinamium (e.g., thiazinamium methylsulfate); thonzylamine hydrochloride; tolpropamine; triprolidine; and tritoqualine.

Antihistamine analogs may also be used in the compositions, methods, and kits of the invention. Antihistamine analogs include 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 1043-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; SCH 37370; SCH 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane.

Other compounds that are suitable for use in the invention are AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that are suitable for use in the invention are described in U.S. Pat. Nos. 2,595,405, 2,709,169, 2,785,202, 2,899,436, 3,014,911, 3,813,384, 3,956,296, 4,254,129, 4,254,130, 4,282,833, 4,283,408, 4,362,736, 4,394,508, 4,285,957, 4,285,958, 4,440,933, 4,510,309, 4,550,116, 4,659,716, 4,692,456, 4,742,175, 4,833,138, 4,908,372, 5,204,249, 5,375,693, 5,578,610, 5,581,011, 5,589,487, 5,663,412, 5,994,549, 6,201,124, and 6,458,958.

Epinastine

In certain embodiments, epinastine or an analog thereof is used in the compositions, methods, and kits of the invention.

Epinastine has the formula:

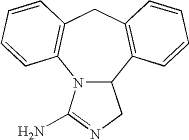

Analogs of epinastine, which are described in U.S. Pat. No. 4,313,931, have the following structure.

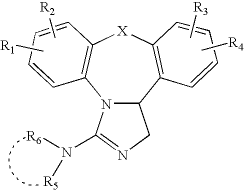

where R₁, R₂, R₃, and R₄, which may be the same or different, each represent a hydrogen or halogen atom or an alkyl or alkoxy group of from 1 to 6 carbon atoms; R₅ and R₆, which may be the same or different, each represent a hydrogen atom, an alkyl group of from 1 to 6 carbon atoms, or an alkenyl group of from 3 to 6 carbon atoms, or R₅ and R₆ together with the nitrogen atom to which they are attached represent a pyrrolidino, piperidino, or morpholino group; and X represents oxygen, sulfur, or a methylene group, and non-toxic, pharmacologically acceptable acid addition salts thereof. These analogs can occur as racemates or as pure enantiomers, or as mixtures with various portions of the enantiomers, each in form of the free bases or the acid addition salts.

Desloratadine

In certain embodiments, desloratadine or a desloratadine analog can be used in the compositions, methods, and kits of the invention. The structure of desloratadine is:

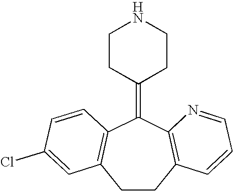

Analogs of desloratadine are described in U.S. Pat. No. 4,659,716 and have the structure:

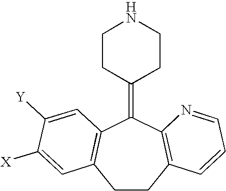

where X and Y independently represent H, halo (i.e., fluoro, chloro, bromo or iodo), or trifluoromethyl with the proviso that at least one of X and Y is halo or trifluoromethyl. Preferred compounds include those where X is F and Y is H or where X is Cl and Y is H.

Related compounds include loratadine, 3-hydroxydesloratadine, des(ethoxycarbonyl)loratadine, and SCH 434. Loratadine functional and/or structural analogs include other H1-receptor antagonists, such as AHR-11325, acrivastine, antazoline, astemizole, azatadine, azelastine, bromopheniramine, carebastine, cetirizine, chlorpheniramine, chlorcyclizine, clemastine, cyproheptadine, descarboethoxyloratadine, dexchlorpheniramine, dimenhydrinate, diphenylpyraline, diphenhydramine, ebastine, fexofenadine, hydroxyzine ketotifen, lodoxamide, levocabastine, methdilazine, mequitazine, oxatomide, pheniramine pyrilamine, promethazine, pyrilamine, setastine, tazifylline, temelastine, terfenadine, trimeprazine, tripelennamine, triprolidine, utrizine, and similar compounds (described, e.g., in U.S. Pat. Nos. 3,956,296, 4,254,129, 4,254,130, 4,283,408, 4,362,736, 4,394,508, 4,285,957, 4,285,958, 4,440,933, 4,510,309, 4,550,116, 4,692,456, 4,742,175, 4,908,372, 5,204,249, 5,375,693, 5,578,610, 5,581,011, 5,589,487, 5,663,412, 5,994,549, 6,201,124, and 6,458,958).

Loratadine, cetirizine, and fexofenadine are second-generation H1-receptor antagonists that lack the sedating effects of many first generation H1-receptor antagonists. Piperidine H1-receptor antagonists include loratadine, cyproheptadine hydrochloride (PERIACTIN), and phenindiamine tartrate (NOLAHIST). piperazine H1-receptor antagonists include hydroxyzine hydrochloride (ATARAX), hydroxyzine pamoate (VISTARIL), cyclizine hydrochloride (MAREZINE), cyclizine lactate, and meclizine hydrochloride.

The structure of loratadine is:

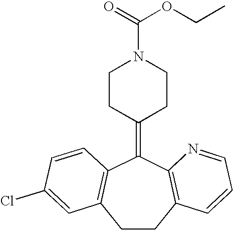

Analogs of loratadine are described in U.S. Pat. No. 4,282,233 and have the structure:

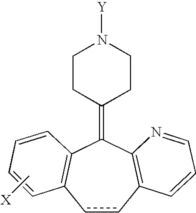

where the dotted line represents an optional double bond and wherein the numbering system used herein is illustrated. In this formula, X is hydrogen or halo and Y is substituted carboxylate or substituted sulfonyl for example Y is —COOR or SO₂R, with the proviso that when Y is —COOR, R is C₁ to C₁₂ alkyl, substituted C₁ to C₁₂ alkyl, phenyl, substituted phenyl, C₇ to C₁₂ phenyl alkyl, C₇ to C₁₂ phenyl alkyl wherein the phenyl moiety is substituted or R is -2, -3, or -4 piperidyl or N-substituted piperidyl wherein the substituents on said substituted C₁ to C₁₂ alkyl are selected from amino or substituted amino and the substituents on said substituted amino are selected from C₁ to C₆ alkyl, the substituents on said substituted phenyl and on said substituted phenyl moiety of the C₇ to C₁₂ phenyl alkyl are selected from C₁ to C₆ alkyl and halo, and the substituent on said N-substituted piperidyl is C₁ to C₄ alkyl; and with the proviso that when Y is SO₂R, R is C₁ to C₁₂ alkyl, phenyl, substituted phenyl, C₇ to C₁₂ phenyl alkyl, C₇ to C₁₂ phenyl alkyl wherein the phenyl moiety is substituted, wherein the substituents on said substituted phenyl and said substituted phenyl moiety of the C₇ to C₁₂ phenyl alkyl are selected from C₁ to C₆ alkyl and halo.

In a preferred embodiment, Y is —COOR and R is C₁ to C₆ alkyl or substituted alkyl, phenyl, substituted phenyl, C₇ to C₁₂ aralkyl or substituted aralkyl or -2, -3 or -4 piperidyl or N-substituted piperidyl. When R is substituted alkyl, R is substituted with amino or with substituted amino. The substituents on the substituted amino are C₁ to C₆ alkyl. The substituents on the aforementioned substituted phenyl and on the phenyl moiety of the substituted aralkyl are preferably C₁ to C₆ alkyl or halo.

In a second preferred embodiment of the present invention, Y is SO₂R and R is C₁ to C₆ alkyl, phenyl, substituted phenyl, C₇ to C₁₂ aralkyl or substituted aralkyl, wherein the substituents on said substituted phenyl and on the phenyl moiety of the substituted aralkyl are C₁ to C₆ alkyl or halo.

The aforementioned alkyl groups may be linear, branched or cyclic or may contain both cyclic and linear or cyclic and branched moieties. Halo may be fluoro, chloro, bromo or iodo.

### Nonsteroidal Immunophilin-Dependent Immunosuppressant

In certain embodiments, a non-steroidal immunophilin-dependent immunosuppressant (NsIDI) is used in the compositions, methods, or kits of the invention. Non-steroidal agents that decreases proinflammatory cytokine production or secretion, binds an immunophilin, or causes a down regulation of the proinflammatory reaction can be used in the invention. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

Cyclosporines

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Pat. Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al. (Antimicrob. Agents Chemother. 44:143-149, 2000).

Tacrolimus

Tacrolimus and tacrolimus analogs are described by Tanaka et al., (J. Am. Chem. Soc., 109:5031, 1987) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus

Pimecrolimus is the 33-epi-chloro derivative of the macrolactam ascomyin. Pimecrolimus structural and functional analogs are described in U.S. Pat. No. 6,384,073.

Rapamycin

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Peptide Moieties

Peptides, peptide mimetics, peptide fragments, either natural, synthetic or chemically modified, that impair the calcineurin-mediated dephosphorylation and nuclear translocation of NFAT are suitable for use in practicing the invention. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell. 1:627-637, 1998). As a class of calcineurin inhibitors, these agents are useful in the methods of the invention.

### Additional Antiviral Agents

One or more (e.g., two, three, four, five, or six) additional antiviral agents can be used in the compositions, methods, and kits of the invention. Exemplary agents are those shown in Table 2. Agents useful in treating viral infections such as influenza include neuraminidase inhibitors (e.g., oseltamivir and zanamivir) and M2 ion channel inhibitors (e.g., amantadine and rimantadine). Other agents which, for example, inhibit viral replication, transcription, reverse transcription, or viral particticle production may also be used in the compositions, methods and kits of the invention.

### Conjugates

If desired, the agents used in any of the combinations described herein may be covalently attached to one another to form a conjugate of formula I.

(A)-(L)-(B) (I)

In formula I, (A) is an agent described herein covalently tethered via a linker (L) to (B), to a second agent described herein.

Conjugates of the invention can be administered to a subject by any route and for the treatment of viral infection (e.g., those described herein such as influenza).

The conjugates of the invention can be prodrugs, releasing drug (A) and drug (B) upon, for example, cleavage of the conjugate by intracellular and extracellular enzymes (e.g., amidases, esterases, and phosphatases). The conjugates of the invention can also be designed to largely remain intact in vivo, resisting cleavage by intracellular and extracellular enzymes. The degradation of the conjugate in vivo can be controlled by the design of linker (L) and the covalent bonds formed with drug (A) and drug (B) during the synthesis of the conjugate.

Conjugates can be prepared using techniques familiar to those skilled in the art. For example, the conjugates can be prepared using the methods disclosed in G. Hermanson, Bioconjugate Techniques, Academic Press, Inc., 1996. The synthesis of conjugates may involve the selective protection and deprotection of alcohols, amines, ketones, sulfhydryls or carboxyl functional groups of drug (A), the linker, and/or drug (B). For example, commonly used protecting groups for amines include carbamates, such as tent-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994. Additional synthetic details are provided below.

### Linkers

The linker component of the invention is, at its simplest, a bond between drug (A) and drug (B), but typically provides a linear, cyclic, or branched molecular skeleton having pendant groups covalently linking drug (A) to drug (B).

Thus, linking of drug (A) to drug (B) is achieved by covalent means, involving bond formation with one or more functional groups located on drug (A) and drug (B). Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of drug (A) and drug (B) may be effected using a linker which contains reactive moieties capable of reaction with such functional groups present in drug (A) and drug (B). For example, an amine group of drug (A) may react with a carboxyl group of the linker, or an activated derivative thereof, resulting in the formation of an amide linking the two.

Examples of moieties capable of reaction with sulfhydryl groups include α-haloacetyl compounds of the type XCH₂CO— (where X=Br, Cl, or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulfide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO— (where X=Br, Cl, or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J. Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g., wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in drug (A) and/or drug (B) may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of drug (A) with a reactive chemical group of drug (B) without introducing additional linking material may, if desired, be used in accordance with the invention.

More commonly, however, the linker will include two or more reactive moieties, as described above, connected by a spacer element. The presence of such a spacer permits bifunctional linkers to react with specific functional groups within drug (A) and drug (B), resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between drug (A) and drug (B).

Spacer elements in the linker typically consist of linear or branched chains and may include a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

In some instances, the linker is described by formula (II):

G¹-(Z¹)ₒ-(Y¹)ᵤ-(Z²)ₛ-(R₃₀)-(Z³)ₜ-(Y²)ᵥ-(Z⁴)ₚ-G² (II)

In formula (II), G¹ is a bond between drug (A) and the linker; G² is a bond between the linker and drug (B); Z′, Z², Z³, and Z⁴ each, independently, is selected from O, S, and NR₃₁; R₃₁ is hydrogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; Y¹ and Y² are each, independently, selected from carbonyl, thiocarbonyl, sulphonyl, or phosphoryl; o, p, s, t, u, and v are each, independently, 0 or 1; and R₃₀ is a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl, or a chemical bond linking G¹-(Z¹)ₒ-(Y¹)ᵤ-(Z²)ₛ- to -(Z³)ₜ-(Y²)ᵥ-(Z⁴)ₚ-G².

Examples of homobifunctional linkers useful in the preparation of conjugates of the invention include, without limitation, diamines and diols selected from ethylenediamine, propylenediamine and hexamethylenediamine, ethylene glycol, diethylene glycol, propylene glycol, 1,4-butanediol, 1,6-hexanediol, cyclohexanediol, and polycaprolactone diol.

### Formulation of Pharmaceutical Compositions

The compositions, methods, and kits of the invention can include formulation(s) of compound(s) that, upon administration to a subject, result in a concentration of the compound(s) that treats a viral infection (e.g., an influenza infection). The compound(s) may be contained in any appropriate amount in any suitable carrier substance, and are generally present in an amount of 1-95% by weight of the total weight of the composition. The composition may be provided in a dosage form that is suitable for the oral, parenteral (e.g., intravenously or intramuscularly), rectal, determatological, cutaneous, nasal, vaginal, inhalant, skin (patch), ocular, intrathecal, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions according to the invention or used in the methods of the invention may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agent(s) of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the combination to a particular target cell type. Administration of compound(s) in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound(s) are formulated with appropriate excipients into a pharmaceutical composition that, upon administration, releases the compound(s) in a controlled manner. Examples include single or multiple unit tablet or capsule compositions, oil solutions, suspensions, emulsions, microcapsules, molecular complexes, microspheres, nanoparticles, patches, and liposomes.

### Delivery of Compound(s)

It is not intended that administration of compounds be limited to a single formulation and delivery method for all compounds of a combination. The combination can be administered using separate formulations and/or delivery methods for each compound of the combination using, for example, any of the above-described formulations and methods. In one example, a first agent is delivered orally, and a second agent is delivered intravenously.

### Dosages

The dosage of a compound or a combination of compounds depends on several factors, including: the administration method, the type of viral infection to be treated, the severity of the infection, whether dosage is designed to treat or prevent a viral infection, and the age, weight, and health of the patient to be treated.

For combinations that include an anti-viral agent in addition to a compound(s) identified herein, the recommended dosage for the anti-viral agent is can be less than or equal to the recommended dose as given in the Physician's Desk Reference, 60^{th} Edition (2006). In other cases, the dosage of the compound or antiviral agent may be higher than the recommended dose.

As described above, the compound in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied. The correct dosage of a compound can be determined by examining the efficacy of the compound in viral replication assays, as well as its toxicity in humans.

An agent is usually given by the same route of administration that is known to be effective for delivering it as a monotherapy. For example, when used in combination therapy an agent is dosed in amounts and frequencies equivalent to or less than those that result in its effective monotherapeutic use.

A combination described herein may be administered to the patient in a single dose or in multiple doses. Components of the combination may be administered separately or together, and by the same or different routes. In addition, various components of the combination may be administered at the same or different times. When multiple doses are administered, the doses may be separated from one another by, for example, one, two, three, four, or five days; one or two weeks; or one month. For example, the combination may be administered once a week for, e.g., 2, 3, 4, 5, 6, 7, 8, 10, 15, 20, or more weeks. Both the frequency of dosing and length of treatment may be different for each compound of the combination. It is to be understood that, for any particular subject, specific dosage regimes should be adjusted over time according to the individual need and the professional judgment of the person administering or supervising the administration of the compositions. For example, the dosage of the combination, or components thereof, can be increased if the lower dose does not sufficiently treat the viral infection. Conversely, the dosage of the combination can be decreased if the viral infection is cleared from the patient.

In other embodiments, agents, either as monotherapies in combination with other agents can be administered at higher dosages than the recommended dosage.

### Example 1

### In-Vitro Activity of Combinations in H5N1 Stimulated Macrophages

Monocytes purified from blood mononuclear cell preparation were differentiated to macrophages (14 days) in 5% autologous serum. Macrophages were then infected with an A/VN/3212/04 (H5N1) virus at a MOI of two. Cells were incubated with the combination, one hour prior to the infection. During the infection, the drug was washed off for 30 minutes and reintroduced for 3 hours. RT-PCR analysis of mRNA in virus infected macrophages was carried out for the following cytokines: TNF-alpha, IFN-beta, IP-10, IL-6, IL-8, H₅N₁ matrix gene (Lee et. al., J. Virol., 79:10147-10154, 2005). Cytotoxicity was evaluated visually and by Beta-actin gene expression. Fifteen combinations of agents were tested at three concentrations each.

From these experiments, the RT-PCR data was analyzed and calculated as a percentage inhibition versus a DMSO-treated control. The percent inhibition data is show in Table 3 below.

TABLE 3

| Test Combination | TNF-α | IFN-β | IP-10 | IL-6 | IL-8 | MCP-1 | M gene |
|---|---|---|---|---|---|---|---|
| Amoxapine 0.3 μM + | ++ | ++ | ++++ | − | + | +++ | + |
| Prednisolone 0.03 μM | | | | | | | |
| Amoxapine 3 μM + | +++ | + | ++ | ++++ | ++ | +++ | − |
| Prednisolone 0.3 μM | | | | | | | |
| Amoxapine 30 μM + | ++++ | +++ | +++++ | +++++ | + | ++++ | − |
| Prednisolone 3 μM | | | | | | | |
| Paroxetine HCl 0.17 μM + | ++ | + | +++ | ++++ | − | ++ | + |
| Prednisolone 0.0062 μM | | | | | | | |
| Paroxetine HCl 1.7 μM + | +++ | + | ++++ | ++++ | + | ++++ | − |
| Prednisolone 0.062 μM | | | | | | | |
| Paroxetine HCl 17 μM + | ++++ | +++ | +++++ | +++++ | − | +++ | + |
| Prednisolone 0.62 μM | | | | | | | |
| Amoxapine 0.2 μM + | − | + | ++ | + | − | − | − |
| Dipyridamole 0.5 μM | | | | | | | |
| Amoxapine 2 μM + | + | + | ++ | − | − | ++ | − |
| Dipyridamole 5 μM | | | | | | | |
| Amoxapine 20 μM + | ++++ | ++++ | ++++ | +++++ | − | ++++ | + |
| Dipyridamole 50 μM | | | | | | | |
| Budesonide 0.00012 μM + | + | + | ++ | ++ | 3 | + | + |
| Nortriptyline HCl | | | | | | | |
| 0.41 μM | | | | | | | |
| Budesonide 0.0012 μM + | ++ | + | +++ | +++++ | +++ | ++++ | − |
| Nortriptyline HCl 4.1 μM | | | | | | | |
| Budesonide 0.012 μM + | +++ | +++ | ++++ | +++++ | − | +++ | + |
| Nortriptyline HCl 41 μM | | | | | | | |
| Dipyridamole 0.0032 μM + | ++ | − | ++++ | +++ | ++ | ++ | − |
| Budesonide 0.0017 | | | | | | | |
| Dipyridamole 0.032 μM + | ++ | − | ++++ | ++++ | + | ++ | − |
| Budesonide 0.017 | | | | | | | |
| Dipyridamole 0.32 μM + | +++ | + | ++++ | ++++ | ++ | +++ | − |
| Budesonide 0.17 | | | | | | | |
| Nortriptyline HCl 0.25 μM + | − | − | ++++ | ++++ | − | − | − |
| Prednisolone 0.062 μM | | | | | | | |
| Nortriptyline HCl 2.5 μM + | ++ | − | ++++ | ++ | − | +++ | − |
| Prednisolone 0.0062 μM | | | | | | | |
| Nortriptyline HCl 25 μM + | ++++ | + | ++++ | ++ | − | +++ | − |
| Prednisolone 0.62 μM | | | | | | | |
| Paroxetine HCl 0.4 μM + | − | − | ++++ | ++ | − | ++ | − |
| Dipyridamole 0.24 μM | | | | | | | |
| Paroxetine HCl 4 μM + | + | + | +++ | ++++ | − | ++ | − |
| Dipyridamole 2.4 μM | | | | | | | |
| Paroxetine HCl 40 μM + | +++ | +++ | +++++ | +++++ | − | + | ++++ |
| Dipyridamole 24 μM | | | | | | | |
| Dipyridamole 0.06 μM + | − | − | ++ | ++ | − | + | − |
| Ibudilast 0.025 μM | | | | | | | |
| Dipyridamole 0.6 μM + | − | − | +++ | − | − | − | − |
| Ibudilast 0.25 μM | | | | | | | |
| Dipyridamole 6 μM + | ++ | + | +++ | ++ | + | ++ | + |
| Ibudilast 2.5 μM | | | | | | | |
| Epinastine 0.22 μM + | + | − | ++++ | +++ | − | + | − |
| Prednisolone 0.0062 μM | | | | | | | |
| Epinastine 2.2 μM + | ++ | − | ++++ | ++++ | ++ | ++ | − |
| Prednisolone 0.062 μM | | | | | | | |
| Epinastine 22 μM + | +++ | ++ | ++++ | + | + | +++ | − |
| Prednisolone 0.62 μM | | | | | | | |
| Bufexamac 0.28 μM + | + | − | +++ | ++ | − | − | − |
| Prednisolone 0.0016 μM | | | | | | | |
| Bufexamac 2.8 μM + | ++ | − | ++++ | +++ | − | ++ | − |
| Prednisolone 0.016 μM | | | | | | | |
| Bufexamac 28 μM + | +++ | + | ++++ | ++++ | ++ | +++ | − |
| Prednisolone 0.16 μM | | | | | | | |
| Sertraline 0.38 μM + | ++ | + | ++ | ++++ | − | ++++ | − |
| Prednisolone 0.025 μM | | | | | | | |
| Sertraline 3.8 μM + | +++ | + | +++++ | +++ | − | − | − |
| Prednisolone 0.25 μM | | | | | | | |
| Sertraline 38 μM + | ++++ | + | +++++ | ++ | − | − | +++++ |
| Prednisolone 2.5 μM | | | | | | | |
| Desloratidine 0.2 μM + | − | − | +++ | +++++ | − | − | − |
| Cyclosporine 0.004 μM | | | | | | | |
| Desloratidine 2 μM + | − | − | +++ | +++ | − | + | − |
| Cyclosporine 0.04 μM | | | | | | | |
| Desloratidine 20 μM + | ++ | + | +++ | ++++ | + | +++ | − |
| Cyclosporine 0.4 μM | | | | | | | |
| CME-Amoxapine 0.17 μM + | + | − | ++++ | − | − | − | − |
| Prednisolone | | | | | | | |
| 0.0063 μM | | | | | | | |
| CME-Amoxapine 1.7 μM + | +++ | + | ++++ | ++++ | + | ++ | − |
| Prednisolone 0.063 μM | | | | | | | |
| CME-Amoxapine 17 μM + | +++ | − | ++++ | + | +++ | +++ | − |
| Prednisolone 0.63 μM | | | | | | | |
| Desloratidine 5.3 μM + | + | − | ++++ | + | − | + | − |
| Nortriptyline HCl 0.73 μM | | | | | | | |
| Desloratidine 16 μM + | +++ | − | +++++ | +++ | − | +++ | − |
| Nortriptyline HCl 2.2 μM | | | | | | | |
| Desloratidine 48 μM + | ++++ | ++ | +++++ | +++ | − | ++ | +++ |
| Nortriptyline HCl 6.6 μM | | | | | | | |
| Desloratidine 5.3 μM + | − | − | ++ | ++ | − | − | − |
| Fluoxetine 0.15 μM | | | | | | | |
| Desloratidine 16 μM + | + | − | ++++ | +++ | − | ++ | − |
| Fluoxetine 0.45 μM | | | | | | | |
| Desloratidine 48 μM + | +++ | ++ | +++++ | ++++ | − | +++ | − |
| Fluoxetine 1.35 μM | | | | | | | |

No inhibition −
0%-20% inhibition +
21%-40% inhibition ++
41%-60% inhibition +++
61%-80% inhibition ++++
81%-100% inhibition +++++

### Example 2

### Activity of Sertraline and Combinations Thereof in Influenza Mouse Model

We also tested the effectiveness of sertraline and combinations thereof in an influenza mouse model. Mouse adapted influenza A/PR/8/34 was procured from American Type Culture Collection (ATCC) and propagated in Madin-Darby Canine Kidney (MDCK) cells. The virus stock was titrated in MDCK cells to give a 10⁸ TCID₅₀/mL, prior to use in mice. The virus stock was diluted in phosphate buffered saline (PBS) such that the working concentration was 10^{4.5} TCID₅₀ of virus per 50 μL.

Specific pathogen free, male C57/BL6 mice weighing 20-25 g were procured from Biological Resource Centre (BRC) and housed in groups of 3, in cages with Corncob bedding (Harlan-Teklad, U.K.). Experiments were conducted in Animal Bio-safety level 3 (ABSL-3) rooms. Cages were placed in isolator maintained at −100 pa pressure and supply of HEPA filtered air. Mice were provided with commercial rodent diet (Harlan-Teklad, U.K.) and distilled water ad libitum.

Mice were orally administered with respective treatments starting 4 hours before virus inoculation daily for five days. At the time of virus inoculation mice were anesthetized with Ketamine (75 mg/kg)+Xylazine (50 mg/kg). 50 μL of 10^{4.5} TCID₅₀ virus suspension was administered intranasally to each mouse. Previous experiments have shown that 10^{4.5} TCID₅₀/mouse of virus is lethal and produces 100% mortality in C57/BL6 mice (data not shown). Mice were weighed daily, and the weights were used for dose adjustment. Sertraline and prednisolone were suspended in 0.5% HPMC and administered once daily while oseltamivir was dissolved in distilled water and administered twice daily. Sertraline, sertraline+prednisolone combination, oseltamivir, and vehicle were orally administered for 5 days starting 4 hr before virus inoculation. The survival rate of animals was monitored for 10 days after infection.

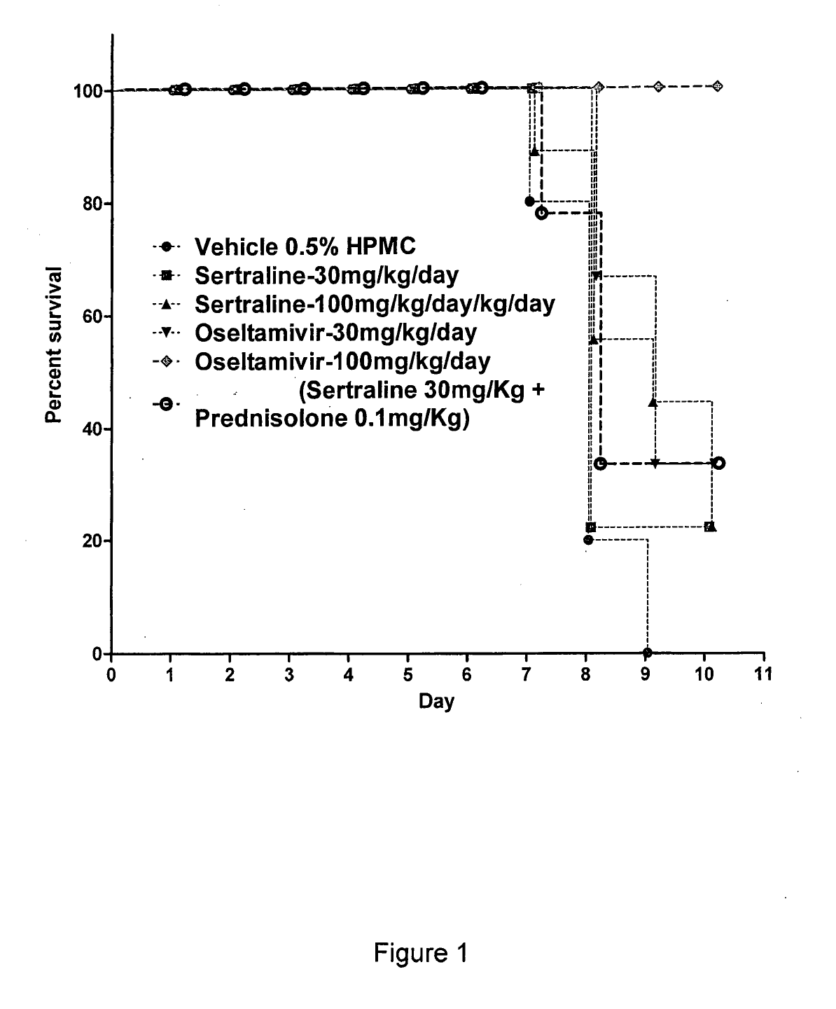

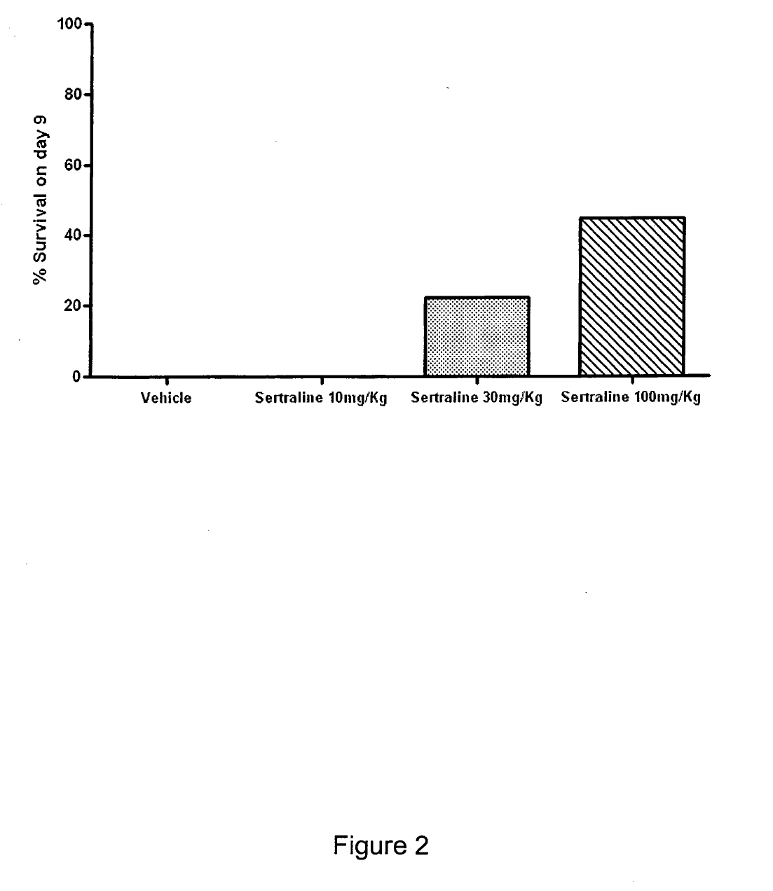

From these experiments, vehicle treated mice began to die on day 7 and their survival rate on day 9 was 0%. The survival rate of mice receiving sertraline at a dose of 30 mg/Kg/day was 22.2% on day 10. In mice treated with sertraline at 100 mg/kg/day, the survival rate was 55.5% on day 8, 44.4% on day 9, and 22.2% on day 10. Thus, sertraline shows dose dependant increase in survival rate by day 9 by which vehicle treated group shows 100% mortality (FIGS. 1 and 2).

Mice treated with a combination of sertraline 30 mg/kg and prednisolone 0.1 mg/Kg showed 30% survival on day 10. Oseltamivir was used as a positive control and the survival rates for 30 mg/kg/day and 100 mg/kg/day were 33.3% and 100% respectively on day 10. Sertraline alone or in combination with prednisolone improves survival rate of C57/BL6 mice infected with lethal dose of influenza A/8/PR/34.

### Other Embodiments

All patents, patent applications including U.S. Provisional Patent Application No. 61/______, filed Mar. 19, 2008, titled “Compositions and Methods for Treatment of Viral Diseases,” Attorney Docket No. 50425/004004, and publications mentioned in this specification are herein incorporated by reference to the same extent as if each independent patent, patent application, or publication was specifically and individually indicated to be incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of molecular biology, medicine, immunology, pharmacology, virology, or related fields are intended to be within the scope of the invention.

What is claimed is:

1. A composition comprising:
(a) a selective serotonin reuptake inhibitor (SSRI); and
(b) an additional antiviral therapy.

2. The composition of claim 1, wherein said SSRI is sertraline, or an analog thereof.

3. The composition of claim 1, wherein said additional antiviral therapy is a Group A antiviral agent.

4. The composition of claim 1, where said additional antiviral agent is oseltamivir, zanamivir, amantadine, or rimantadine.

5. The composition of claim 1, wherein (a) and (b) are present in amounts that together are effective to treat or prevent a viral infection.

6. The composition of claim 1, wherein said viral infection is caused by an influenza virus.

7. A composition comprising:
(a) a combination of agents selected from the combinations of Table 1; and
(b) an additional antiviral therapy.

8. The composition of claim 7, wherein said additional antiviral therapy is a Group A antiviral agent.

9. The composition of claim 7, where said additional antiviral agent is oseltamivir, zanamivir, amantadine, or rimantadine.

10. The composition of claim 7, wherein (a) and (b) are present in amounts that together are effective to treat or prevent a viral infection.

11. The composition of claim 7, wherein said viral infection is caused by an influenza virus.

12. A method for treating or preventing a viral infection in a patient, said method comprising administering to said subject an amount of an SSRI sufficient to treat or prevent said viral infection in said patient.

13. The method of claim 12, wherein said SSRI is sertraline, or an analog thereof.

14. The method of claim 12, wherein said viral infection is an influenza virus infection.

15. The method of claim 12, wherein said method further comprises administration of an additional antiviral therapy.

16. The method of claim 15, wherein said additional antiviral therapy is a Group A antiviral.

17. A method for treating or preventing a viral infection in a patient, said method comprising administering a pair of agents selected from the group consisting of (a) a tricyclic antidepressant and a corticosteroid, (b) an SSRI and a corticosteroid, (c) a tricyclic antidepressant and a tetra-substituted pyrimidopyrimidine, (d) corticosteroid and a tetra-substituted pyrimidopyrimidine, (e) an SSRI and a tetra-substituted pyrimidopyrimidine, (f) a tetra-substituted pyrimidopyrimidine and ibudilast, (g) an antihistamine and a corticosteroid, (h) a corticosteroid and bufexamac, (i) an antihistamine and a non-steroidal immunophilin-dependent immunosuppressant (NsIDI), (j) a tricyclic antidepressant and an antihistamine, and (k) an antihistamine and an SSRI, wherein said pair of agents is administered simultaneously or within 14 days of each other in amounts that together are sufficient to treat or prevent said viral infection in said patient.

18. The method of claim 17, wherein said viral infection is an influenza virus infection.

19. The method of claim 17, wherein said method further comprises administration of an additional antiviral therapy.

20. The method of claim 19, wherein said additional antiviral therapy is a Group A antiviral.

21. A method for treating or preventing a viral infection in a patient, said method comprising administering to said patient a combination of agents of Table 1, wherein each agent of said combination is administered simultaneously or within 14 days of each other in amounts that together are effective to treat or prevent said viral infection in said patient.

22. The method of claim 21, wherein said viral infection is an influenza virus infection.

23. The method of claim 21, wherein said method further comprises administration of an additional antiviral therapy.

24. The method of claim 23, wherein said additional antiviral therapy is a Group A antiviral.
